(19) United States
(12) Patent Application Publication
(10) Pub. No.: US 2026/0284349 A1
(43) Pub. Date: Sep. 24, 2026

(54) A SCENT DELIVERY DEVICE AND REPLACEABLE SCENT CARTRIDGES FOR USE THEREIN

(71) Applicant: SCENTIENT LTD, London (GB)

(72) Inventors: Ivan Novikov, London (GB); Anastasia Georgievskaya, London (GB)

(21) Appl. No.: 19/479,273

(22) PCT Filed: Apr. 25, 2024
(86) PCT No.: PCT/GB2024/051080

§ 371 Date: Oct. 27, 2025

(30) Foreign Application Priority Data
Apr. 28, 2023 (GB) 2306389.4

(51) Int. Cl. A61M 21/00; A61B 5/00; B05B 17/06; F24F 8/50

(52) U.S. Cl. CPC A61M 21/00; A61B 5/4011; B05B 17/0607; F24F 8/50; A61M 2021/0016

(57) ABSTRACT

A scent delivery device and replaceable scent cartridges for use therein A replaceable scent cartridge for a scent delivery device is provided comprising: a container configured to reversibly couple to a piezoelectric element configured to atomise the scented media; and a porous material configured to carry a scented media, arranged at least partially inside the container, wherein the porous material is configured to contact a surface of the piezoelectric element when the container is coupled to the piezoelectric element. A resealable scent cartridge for a scent delivery device is also provided comprising a container configured to hold a scented media, and an impermeable membrane comprising at least one resealable aperture, wherein the impermeable membrane is configured to seal the resealable aperture in a closed configuration, and unseal the resealable aperture in an open configuration. A wearable scent delivery device is also provided comprising a wearable housing configured to be worn by a user, and at least one atomiser element configured to couple to a scent cartridge and atomise scented media within the scent cartridge. The housing also comprises at least one outlet, wherein each atomiser element is in fluid communication with at least one outlet such that scented media atomised by the at least one atomiser element is configured to leave the housing via the outlet.

### FIELD OF THE INVENTION

The present disclosure relates to a scent delivery device and replaceable scent cartridge for use therewith, in particular a wearable scent delivery device.

### BACKGROUND

Olfaction is an important and powerful human sense which strongly impacts how humans perceive their environment. Despite this, existing extended reality experiences, including virtual reality (VR) and augmented reality (AR), primarily rely on means of visual and auditory stimulation. While introduction of scent into the VR environment has been suggested, the successful integration of scent in VR, as well as other extended reality applications, has been limited.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

### Scent Cartridges

In a first aspect of the invention, there is provided a replaceable scent cartridge for a scent delivery device. The scent cartridge comprises a container configured to reversibly couple to a piezoelectric element, and a porous material arranged at least partially inside the container, wherein the porous material is configured to carry a scented media. The porous material is configured to contact a surface of the piezoelectric element when the container is coupled to the piezoelectric element, wherein the piezoelectric element is configured to atomise the scented media. The contact of the porous material may be advantageous to transport the scented media to the piezoelectric element for atomisation. This may reduce blockages within the cartridge caused by the scented media, as the scented media is carried by the porous material. Furthermore, the porous material may help to control the delivery of the scented media to the piezoelectric element throughout the life of the cartridge. This configuration may also reduce the pressure on the piezoelectric element compared to configurations using liquid scented media only, wherein the liquid directly contacts the piezoelectric element. Furthermore, the porous material configuration may be configured to reduce damage and degradation of the piezoelectric element in use, compared to configurations using liquid scented media only. This may be advantageous to extend the useable lifetime of a piezoelectric element. Configurations reliant on a column of liquid contacting the piezoelectric element can damage the piezoelectric element, in particular when the piezoelectric element continues to vibrate after the column of liquid empties and the piezoelectric element is left dry. The provision of the porous material which is configured to hold scented liquid therefore avoids this situation wherein the piezoelectric element continues to vibrate even after the column of liquid has been atomised, and as such, damage to the piezoelectric element may be reduced.

Optionally, the cartridge may further comprise said piezoelectric element.

The container may be configured to reversibly couple to a lid portion, wherein the piezoelectric element is arranged within the lid portion. This may be advantageous to allow the piezoelectric element to be reversibly coupled to the container in a controlled manner. For example, the container may be configured to reversibly couple to a lid portion via a screw-closure, snap-fit closure, or any other suitable lid closure. The lid comprising the piezoelectric element may be configured to be reversibly coupled to a plurality of replaceable cartridges, such that the same piezoelectric element can be used without replacement as the piezoelectric element has a longer usable life compared to a replaceable cartridge which may contain a finite supply of scented media. As such, this may reduce waste, in particular reducing waste of electrical and/or electronic components, such as the piezoelectric element.

The container may further comprise a biasing means, wherein the biasing means is configured to bias the porous material towards the piezoelectric element. This may be advantageous to provide consistent contact between the porous material and the piezoelectric element, for example to improve the supply of scented liquid to the piezoelectric element. In some examples, the biasing means may be a spring, such as, but not limited to, an ortho-planar spring or a conical spring.

The container may comprise a reservoir configured to hold scented liquid, wherein the porous material is arranged between the reservoir and the piezoelectric element, and wherein the porous material is configured to carry the scented liquid. For example, the porous material may be arranged in a layer of porous material between the reservoir and the piezoelectric element. Alternatively, the porous material may comprise a wick element arranged between the reservoir and the piezoelectric element.

In some examples, the container comprises a cavity, wherein the cavity comprises the porous material soaked in scented liquid. The cavity may comprise only the porous material soaked in scented liquid. In such examples, the cavity may not comprise a volume of scented liquid per se. This may reduce leakages of scented liquid from the cartridge. This configuration may also reduce the pressure on the piezoelectric element compared to configurations comprising volumes of liquid scented media, wherein the liquid directly contacts the piezoelectric element.

The porous material may be configured to transport scented media to the piezoelectric element by capillary action. This may be advantageous as the porous material is not reliant on gravity. As such, the porous material may still transport scented media to the piezoelectric element when the container is arranged below the piezoelectric element in use, for example. This may also be advantageous such that scented liquid does not block the piezoelectric element, whilst also relieving pressure on piezoelectric element. This may also reduce leakages of scented liquid.

The piezoelectric element may be a microporous piezoelectric element. This may be advantageous such that the atomised scented media is released through the pores in the microporous piezoelectric element.

In some examples, the replaceable scent cartridge may further comprise a lid configured to be coupled to the container, wherein the lid comprises the piezoelectric element.

The porous material may comprise a fibrous material, however the skilled person will understand that any other porous material may be used. In some examples, the porous material is highly absorbent of the scented media.

The scent cartridge may comprise the scented media. Optionally, the scent cartridge further comprises a scent indication, wherein the scent indication comprises a set of parameters for atomisation of the scented media contained within the cartridge. For example, the scent indication may comprise a scent label, such as a QR code, or other encoded indication associated with the scented media contained within the cartridge. The set of parameters for atomisation may include a delivery pattern for atomisation. For example, for a scented media configured to be atomised by a piezoelectric element, the set of parameters may include at least one of (i) an optimum duration, or range of duration, of vibration of a piezoelectric element, and (ii) an optimum frequency, or range of frequencies, of vibration of a piezoelectric element, suitable for atomising the particular scented media.

However, the skilled person will understand that the scent indication is not intended to be limited to a scent label, and that other scent indication means may be used. For example, in another example, the scent cartridge may comprise at least one electrical contact configured to provide a scent indication. For example, the scent cartridge may comprise a pogo pin, or other electrical contact, configured to electrically contact a corresponding detection contact of the scent delivery device when the scent cartridge is installed within the scent delivery device. Upon electrical connection between the cartridge's scent indication contact and the delivery device's corresponding electrical contact, a microcontroller installed within the scent cartridge may be configured to send a signal comprising the scent indication (e.g. scent ID) and/or the set of parameters for atomisation to the scent delivery device, via the electrical coupling between the cartridge's electrical contact and the device electrical contact.

In another example, the scent cartridge may comprise an electrical contact, such as but not limited to a pogo pin, coupled to a resistor, wherein the electrical contact is configured to electrically contact a corresponding detection contact of the scent delivery device when the scent cartridge is installed within the scent delivery device. Upon electrical connection between the cartridge contact and the detection contact, the scent delivery device may be configured to infer the scent indication (e.g. scent ID) and/or the set of parameters for atomisation based on measuring the resistance value of the cartridge resistor, via the electrical connection between the cartridge electrical contact and the device detection contact.

In another example, the scent cartridge may also comprise a plurality of electrical contacts, such as but not limited to a plurality of pogo pins. Each electrical contact on the cartridge may represent one bit in a scent ID. For example, cartridges comprising 8 electrical contacts may comprise an eight-digit binary scent ID. The plurality of electrical contacts of the scent cartridge may be configured to electrically contact the plurality of detection contacts in the scent delivery device when the scent cartridge is installed within the scent delivery device. Upon electrical connection between the cartridge contact and the detection contact, the scent delivery device is configured to identify the scent cartridge by reading each of the pins which together provide a binary scent ID (e.g. “10011011”). The set of parameters for atomisation may be inferred by comparison of the binary scent ID with a lookup table. Alternatively, each scent cartridge may have a specific shape (and/or a specific arrangement of electrical contacts) such that only a portion of electrical contacts form an electrical connection with the scent delivery device; and wherein, when inserted into the scent delivery device, the scent delivery device is configured to infer a scent ID of the cartridge based on the specific number and/or arrangement of electrical contacts in contact with the scent delivery device.

The container may further comprise a cleaning means, wherein the cleaning means is configured to clean the piezoelectric element, for example when the container is coupled to the piezoelectric element in use. The cleaning means may comprise a cleaning cavity configured to hold cleaning fluid, wherein the cleaning cavity is configured to release cleaning fluid to clean the piezoelectric element. For example, the cleaning means may be configured to clean the piezoelectric element upon being coupled to the container. This may be advantageous to clean away any residual scented media on the piezoelectric element, for example from a previous cartridge.

The cartridge may further comprise a sensor configured to detect an indication of the amount of scented media within the container. This may be advantageous to alert a user when a cartridge needs to be replaced or refilled, or when a cartridge will soon need to be replaced or refilled. This may also be advantageous to reduce damage and degradation of the piezoelectric element in use, wherein the piezoelectric element can be damaged when it continues to actuate after the scented media has been fully atomised and the piezoelectric element is left dry. Amount may be quantified by, but is not limited to, at least one of volume of scented media within the container, weight of scented media within the container, moisture content of the porous media configured to hold the scented media, and/or fluid level of scented media within the container. For example, the sensor may be configured to detect when the amount of scented media within the container is below a predetermined threshold, for example when the liquid level of scented liquid is below a predetermined threshold. Alternatively or in addition, the sensor may be configured to detect concentration of the scented media released from the replaceable cartridge during atomisation, for example but not limited to using a gas sensor or flowmeter. Alternatively or in addition, the sensor may be configured for at least one of: (i) capacitive moisture sensing, wherein the sensor is configured to detect changes in capacitance due to changes in the dielectric properties of a material, such as the porous material, caused by moisture; (ii) resistive moisture sensing, wherein the sensor is configured to detect changes in resistance due to changes in the electrical conductivity of a material, such as the porous material, caused by moisture; and (iii) surface moisture measurement (non-contact), wherein the sensor is configured to Time Domain Reflectometry (TDR) or Near-Infrared Reflectance (NIR).

Alternatively, or in addition, the sensor may be configured to detect an indication of the amount of scented media within the container by measuring total atomisation time for the cartridge during its lifetime, and comparing this to a pre-determined maximum total atomisation time for the cartridge. However, it is noted that this method does not account for potential evaporation during periods of non-use. As such, in some examples, the sensor may assume a base level of evaporation based on the age of the cartridge.

The cartridge may comprise a communications interface, wherein the communications interface is configured to send a signal to the piezoelectric element to stop actuation of piezoelectric element in the event that the indication of the amount of scented media within the container, detected by the sensor, is below a predetermined threshold. This may be advantageous to reduce damage and degradation of the piezoelectric element in use, wherein the piezoelectric element can be damaged if it continues to actuate after the scented media has been fully atomised.

The cartridge may comprise a nozzle configured to be in fluid communication with the piezoelectric element, wherein the nozzle is configured to provide an outlet for scented media atomised by the piezoelectric element. The nozzle may be configured to protrude from an outlet of a scent delivery device, when the replaceable scent cartridge is installed within said scent delivery device in use. This may be advantageous to ensure that all atomised scent particles are dispensed outside of the device, preventing atomised scent particles becoming trapped within the scent delivery device or cartridge housing.

The cartridge may further comprise at least one electrical contact, wherein the at least one electrical contact is configured electrically couple the piezoelectric element to an electrical contact of a scent delivery device when the replaceable scent cartridge is installed within said scent delivery device. This may be advantageous to allow the scent delivery device to power and control operation of the piezoelectric element within the cartridge, reducing the control electronics required by the cartridge. As an example, the cartridge may comprise a set of pogo pins as the electrical contact. The electrical contact of the cartridge may be configured to be electrically coupled to the electrical contact of a scent delivery device cartridge when the cartridge is retained with the scent delivery device housing, for example by a snap-or push-fit, or by magnetic attraction between magnets arranged on both the cartridge and within the device housing. However, the skilled person will understand that any other suitable methods for retaining a cartridge within a device, or for facilitating an electrical coupling, may be used.

In another aspect of the invention, there is provided a resealable scent cartridge for a scent delivery device. The resealable scent cartridge comprises a container configured to hold a scented media, and an impermeable membrane comprising at least one resealable aperture, wherein the impermeable membrane is configured to seal the resealable aperture in a closed configuration, and unseal the resealable aperture in an open configuration. The impermeable membrane is also configured to seal the container in the closed configuration. The resealable cartridge may be advantageous to reduce leakage of scented media from the container, in particular during installation or removal of the scent cartridge within a scent delivery device. This may also be advantageous to allow scent cartridges to be interchanged and reused within a scent delivery device, reducing wastage of scented media and cartridges. In particular, this may permit removal and reuse of cartridge which still contain scented media, rather than only removing and discarding scent cartridges once emptied of scented media. For example, in use, a first resealable scent cartridge comprising a first scent may be interchanged within a scent delivery device for a second resealable scent cartridge comprising a second scent, however as the first scent cartridge is resealable this allows it to be retained and reused in future, rather than being thrown away or otherwise being unable to change scented media within the scent delivery device until the first scent cartridge has run out of scented media.

The impermeable membrane may be configured to seal the container, for example such that scented media is configured to be sealed within the container in the closed configuration, and scented media is configured to leave the container via the resealable aperture in the open configuration.

In some examples, it is envisaged that the scented media may be a scented liquid. In such examples, the resealable cartridge may be advantageous to reduce leakage of scented liquid from the container.

The resealable scent cartridge may be configured to be biased into the open configuration by deforming the impermeable membrane. For example, cartridge may be configured to deform the impermeable membrane into the open configuration when installed within a scent delivery device. This may be advantageous to ensure that scented media is able to be released from the container when the cartridge is installed within a scent delivery device in use.

In some examples, the impermeable membrane may be configured to be deformed by deforming the container, for example wherein the container may be configured to be deformed when installed within a scent delivery device.

The cartridge may be configured to couple to a lid, wherein the impermeable membrane is configured to be biased into the open configuration when the cartridge is coupled to the lid. In some examples, the lid may comprise an atomiser element configured to atomise scented media, such as a piezoelectric element.

The lid may be configured to deform the container such that the impermeable membrane is deformed into the open configuration.

Alternatively, or in addition, the impermeable membrane may be configured to be biased into the open configuration by a structure coupled to a surface of the lid. In some examples, the structure is configured to extend through the at least one resealable aperture.

The scent cartridge may comprise the scented media. Optionally, the scent cartridge further comprises a scent indication, wherein the scent indication comprises a set of parameters for atomisation of the scented media contained within the cartridge. For example, the scent indication may comprise a scent label, such as a QR code, or other encoded indication associated with the scented media contained within the cartridge. The set of parameters for atomisation may include a delivery pattern for atomisation. For example, for a scented media configured to be atomised by a piezoelectric element, the set of parameters may include at least one of (i) an optimum duration, or range of duration, of vibration of a piezoelectric element, and (ii) an optimum frequency, or range of frequencies, of vibration of a piezoelectric element, suitable for atomising the particular scented media.

The container may further comprise a cleaning means, wherein the cleaning means is configured to clean the piezoelectric element, for example when the container is coupled to the piezoelectric element in use. The cleaning means may comprise a cleaning cavity configured to hold cleaning fluid, wherein the cleaning cavity is configured to release cleaning fluid to clean the piezoelectric element. For example, the cleaning means may be configured to clean the piezoelectric element upon being coupled to the container. This may be advantageous to clean away any residual scented media on the piezoelectric element, for example from a previous cartridge.

The cartridge may further comprise a sensor configured to detect an indication of the amount of scented media within the container. This may be advantageous to alert a user when a cartridge needs to be replaced or refilled, or when a cartridge will soon need to be replaced or refilled. For example, the sensor may be configured to detect when the amount of scented media within the container is below a predetermined threshold. Amount may be quantified by, but is not limited to, at least one of volume of scented media within the container, weight of scented media within the container, and/or fluid level of scented media within the container. Alternatively or in addition, the sensor may be configured to detect concentration of the scented media released from the replaceable cartridge during atomisation, for example using a gas sensor or similar. Alternatively or in addition, the sensor may be configured to detect gas flow of the scented media released from the replaceable cartridge during atomisation, for example using a flowmeter.

In another aspect of the invention, there is provided a scent delivery device configured for use with any of the scent cartridges disclosed herein. For example, the scent delivery device may be configured to couple to at least one scent cartridge disclosed herein, and wherein the scent delivery device is configured to atomise scented media held within said scent cartridge.

### Scent Delivery Device

In another aspect of the invention, a scent delivery device is provided comprising at least one scent cartridge of any preceding aspect of the invention. For example, the scent delivery device may comprise a wearable housing configured to be worn by a user, wherein the housing comprises at least one outlet, the at least one scent cartridge in fluid communication with at least one outlet such that atomised scented media is configured to leave the housing via the outlet, and a controller configured to control a piezoelectric element to control atomisation of scented media held by said scent cartridge.

In another aspect of the invention, there is provided a scent delivery device comprising a wearable housing configured to be worn by a user, at least one cartridge arranged within the housing, the cartridge being configured to hold a scented media; and at least one atomiser element. Each atomiser element is configured to couple to a cartridge, wherein the atomiser element is configured to atomise scented media held by the cartridge. The housing further comprises at least one outlet, wherein each cartridge is in fluid communication with at least one outlet such that the atomised scented media is configured to leave the housing via the outlet.

Alternatively, the at least one atomiser element, such as a piezoelectric element, may be provided within the cartridge itself. In such examples, a scent delivery device may comprise a wearable housing configured to be worn by a user, wherein the housing comprises at least one outlet; and at least one cartridge receiving portion configured to receive a scent cartridge, wherein the at least one cartridge receiving portion comprises an electrical contact configured to couple to an atomiser element housed within said scent cartridge when said scent cartridge is arranged within the cartridge receiving portion; and a controller, coupled to the electrical contact, the controller being configured to control said atomiser element of said scent cartridge, via the electrical contact, to control atomisation of scented media held by said scent cartridge; wherein the at least one cartridge receiving portion is in fluid communication with at least one outlet such that scented media atomised by said scent cartridge is configured to leave the housing via the outlet.

Existing scent delivery devices typically attach to a virtual reality (VR) headset, and thus do not comprise a wearable housing configured to be worn by a user. Whilst this may be suitable for use with immersive virtual reality experiences, including the use of a virtual reality (VR) headset, these existing devices are not suitable for other olfactory extended reality experiences which do not use a virtual reality headset.

In contrast, the wearable scent delivery device of the present invention may be suitable for a multitude of olfactory extended reality experiences (including but not limited to augmented reality experiences). One example use may be for occupational training, for example wherein a user is required to identify and react to different smells, for example emergency personnel reacting to the smell of burning. In dangerous situations humans tend to revert to their basic senses, with olfaction being vital when it comes to important, quick, and instinctive decisions. Trained personnel can use scents to gather important information such as identifying what kind of, potentially toxic, fuel is burning, detecting gas leaks, and other environmental clues. The device disclosed herein may be used for training emergency and medical personnel, helping to vastly increase the number of scenarios which can be replicated during training, while improving safety through using simulated environments with enhanced realism by adding safe synthetic scents.

Another example use may be medical diagnosis, monitoring, research, and/or treatment, for example for anosmia (loss of sense of smell), as well as other therapeutic uses, for example for therapies for patients with memory loss or dementia which may rely on the delivery of smells to trigger memories.

A further example use may be for entertainment purposes, for example to provide immersive experiences at cinemas, art galleries, exhibitions, or in other entertainment applications. However, the skilled person will understand that these are only a few example applications, and the device may be used for any other personal scent delivery application. The skilled person will also understand that the wearable scent delivery device of the present invention may also be for suitable for use in immersive virtual reality (VR) experiences, for example using a VR headset. However, the wearable scent delivery device may be advantageous over a device which attaches to a VR headset directly as such devices may be configured for use with one or a selection of specific VR headset models, whereas the wearable scent delivery device is agnostic to the model or brand of VR device as the wearable device is configured to be worn by the user, and therefore does not require special mounting to attach onto a VR headset.

In some examples, the wearable housing may comprise a neck-mountable housing configured to be worn at least partially around the neck of a user. This may be advantageous as the neck-mountable housing allows the device to be positioned in proximity to a user's head and nose for optimum scent delivery to a user, whilst also avoiding potentially cumbersome, uncomfortable, and intrusive attachment to a user's head or face. A neck-mountable housing may also be advantageous as variation in human neck size is minimal relative to other parts of the human body, this means a device of a given size is likely to fit a variety of users of different sizes.

The housing may comprise an arched shape configured to be worn around the neck of a user. In some examples, the arched shape housing may comprise an outlet at each end of the arched shape. The skilled person will understand that the outlets are not to be limited to being positioned solely at the terminal end of housing, rather wherein the arched shape housing comprises an outlet at each end may be include any configuration wherein outlets are arranged either side of the mid-point of the arch shaped housing. This may be advantageous to provide stereo scent delivery, for example to provide a user with a sense of directional smell (e.g., by stereo olfaction).

The skilled person will understand that the provision of stereo scent delivery is not limited to an arched shape housing. Rather, any shaped housing may comprise at least two outlets arranged on opposing portions of the housing, configured for stereo scent delivery. Preferably, opposing outlets configured for stereo scent delivery are configured to be arranged on either side of sagittal plane of a user when in use. A pair of opposing stereo outlets may be fluidically isolated from tone another within the housing, wherein each outlet is coupled to a respective cartridge and atomiser element configured for independent control.

Alternatively or in addition, the outlets are configured to be positioned on or forwards of the frontal (or coronal) plane of a user in use, wherein forwards of the frontal plane corresponds to the anterior direction. This may be advantageous to effectively deliver the released scents in the proximity of a user's nose.

In some examples, at least one outlet may be positioned on the housing such that it is configured to be arranged in the posterior direction relative to the frontal (or coronal) plane of a user in use. This may be advantageous to provide a sense of directional smell.

The at least one atomiser element may comprise at least one piezoelectric element. Preferably, a microporous piezoelectric element.

The wearable housing may be configured to attach to a user. Alternatively, or in addition, the wearable housing may be configured to attach to an item of clothing worn by the user. For example, the wearable housing may be configured to attach to a collar or neckline of an item of clothing, however the skilled person will understand that this is merely an example, and the wearable housing may be configured to attach to any item of clothing in any suitable way. In some examples, the wearable housing may comprise a clip, pin, or other fastening means configured to attach the device to an item of clothing.

The housing may further comprise at least one flexible portion configured to be arranged adjacent to the back of the neck of a user. This may be advantageous for ease of donning the wearable device, for example wherein the housing may be deformed adjacent to the back of the neck of a user to accommodate for different neck sizes.

The housing may further comprise a moveable portion configured to be repositioned relative to the user, wherein the outlet is arranged within the moveable portion. This may be advantageous such that the position of the outlets may be adjusted based on the user, for example such that the outlets may be repositioned to direct scented media from the outlets towards the nose of the user.

The scent delivery device may further comprise at least one fan. In some examples, the fan may be configured to clear the atomised scented media. This may be advantageous to clear lingering scents. In some examples, the fan may be configured to clear the atomised scented media by drawing air into the housing and through a filter. The fan may be configured to operate after the atomiser element. Providing a fan and filter may be additionally advantageous to provide distributed air filtering within an environment or space comprising many scent delivery devices.

Each atomiser element may be configured to be independently controlled. This may be advantageous to allow a single device to house a plurality of cartridges comprising different scented media, wherein each scent may be delivered independently via independent control of the associated atomiser element. This may also be advantageous to provide a sense of stereo olfaction, for example by controlling one atomiser element coupled to an outlet at one end of the housing, independently from a second atomiser element coupled to a second outlet at another end of the housing.

Alternatively, or in addition, the device may be configured to atomise and release more than one scent simultaneously, from a plurality of cartridges. This may be advantageous to render a complex scent.

The scent delivery device may further comprise a wireless communications interface configured to receive control signals from a remote device, wherein each atomiser element is configured to be controlled based on control signals received from a remote device via the wireless communications interface. Example remote devices may include, but are not limited to, a VR headset or device, a user computing device, such as a smart phone, or any other remote computing device. This may be advantageous to allow the control of the device to be synced according to other external media or devices.

The scent delivery device may further comprise an inertial measurement unit (IMU), wherein each atomiser element is configured to be controlled based on an indication determined by the inertial measurement unit. This may be advantageous for scent delivery applications based on movement of the user. For example, the IMU may comprise at least one of an accelerometer, gyroscope, and/or magnetometer.

The scent delivery device may further comprise a means for determining location, wherein each atomiser element is configured to be controlled based on an indication determined by the means for determining location. This may be advantageous for scent delivery applications based on movement of the user. For example, the means for determining location may comprise a GPS receiver, Bluetooth receiver, or any other suitable means. The scent delivery device may further comprise at least one cleaning means, wherein each cleaning means is configured to clean the at least one atomiser element. The cleaning means may comprise at least one cleaning cavity configured to hold cleaning fluid, wherein the cleaning cavity is configured to release cleaning fluid to clean at least one atomiser element. This may be advantageous to clean away any residual scented media on the piezoelectric element, for example from a previous use or previous cartridge.

The at least one cartridge may be configured to be replaceable. Optionally, the scent delivery device may further comprise at least one cartridge sensor, each cartridge sensor being configured to detect an indication of the amount of scented media within at least one replaceable cartridge arranged within the housing. This may be advantageous to alert a user when a cartridge needs to be replaced or refilled, or when a cartridge will soon need to be replaced or refilled. Amount may be quantified by, but is not limited to, at least one of volume of scented media within the container, weight of scented media within the container, moisture content of the porous media configured to hold the scented media, and/or fluid level of scented media within the container. For example, the sensor may be configured to detect when the amount of scented media within the container is below a predetermined threshold, for example when the liquid level of scented liquid is below a predetermined threshold. Alternatively or in addition, the sensor may be configured to detect concentration of the scented media released from the replaceable cartridge during atomisation, for example but not limited to using a gas sensor or flowmeter.

Alternatively or in addition, the sensor may be configured to sense moisture content of the porous material, for example wherein the sensor may be configured for at least one of: (i) capacitive moisture sensing, wherein the sensor is configured to detect changes in capacitance due to changes in the dielectric properties of a material, such as the porous material, caused by moisture; (ii) resistive moisture sensing, wherein the sensor is configured to detect changes in resistance due to changes in the electrical conductivity of a material, such as the porous material, caused by moisture; and (iii) surface moisture measurement (non-contact), wherein the sensor is configured to Time Domain Reflectometry (TDR) or Near-Infrared Reflectance (NIR).

Optionally, the cartridge sensor may be coupled to a protruding structure, wherein the protruding structure is configured to extend into a cartridge, for example through the porous material or a resealable aperture of the cartridge.

Alternatively, or in addition, the sensor may be configured to detect an indication of the amount of scented media within the container by measuring total atomisation time for the cartridge during its lifetime, and comparing this to a pre-determined maximum total atomisation time for the cartridge. However, it is noted that this method does not account for potential evaporation during periods of non-use. As such, in some examples, the sensor may assume a base level of evaporation based on the age of the cartridge.

The at least one cartridge sensor may be configured to send a signal to the atomiser element, such as a piezoelectric element, to stop actuation of the atomiser element in the event that the indication of the amount of scented media within the container, detected by the cartridge sensor, is below a predetermined threshold. This may be advantageous to reduce damage and degradation of the atomiser element in use, wherein the atomiser element, in particular piezoelectric elements, can be damaged if they continue to actuate after the scented media has been fully atomised.

For example, the cartridge sensor may be configured to detect when the amount of scented media within the replaceable cartridge is below a predetermined threshold.

Wherein each cartridge is configured to be replaceable, optionally each replaceable cartridge may comprise a scent indication, wherein the scent indication comprises a set of parameters for atomisation of the scented media. In such examples, the scent delivery device may further comprise a detection means configured to detect the scent indication, and a processor configured to control the atomiser element based on the detected scented indication. For example, the scent indication may comprise a scent label, such as a QR code, or other encoded indication associated with the scented media contained within the cartridge. The set of parameters for atomisation may include a delivery pattern for atomisation. For example, for a scented media configured to be atomised by a piezoelectric element, the set of parameters may include at least one of (i) an optimum duration, or range of duration, of vibration of a piezoelectric element, (ii) an optimum frequency, or range of frequencies, of vibration of a piezoelectric element, and (iii) an optimum pulse width modulation (PWM) pattern, suitable for atomising the particular scented media. In one example, the scent delivery device may comprise a detection means configured to scan the QR code to retrieve the set of parameters, and a processor configured to control the piezoelectric element based on the set of parameters retrieved by the detection means. However, the skilled person will understand that the detection means is not intended to be limited to a detection means configured to scan a QR code. For example, in another example, the scent delivery device may comprise a detection means, such as an electrical contact, configured to be electrically connected to a scent cartridge installed within the device. The detection means may be configured to retrieve the scent indication and/or the set of parameters for atomisation via the electrical contact. For example, the scent cartridge may comprise a pogo pin, or other electrical contact, configured to electrically contact a corresponding detection contact of the scent delivery device when the scent cartridge is installed within the scent delivery device. Upon electrical connection between the cartridge contact and the detection contact, the scent cartridge may be configured to send a signal comprising the scent indication (e.g. scent ID) and/or the set of parameters for atomisation to the scent delivery device, via the electrical coupling between the cartridge's electrical contact and the detection contact.

In another example, the scent delivery device may comprise a detection means comprising a detection electrical contact, such as a pogo pin, and a means for measuring electrical resistance coupled to the electrical contact. In such examples, the scent cartridge may comprise an electrical contact, such as a pogo pin, coupled to a resistor, wherein the electrical contact is configured to electrically contact the detection contact of the scent delivery device when the scent cartridge is installed within the scent delivery device. Upon electrical connection between the cartridge contact and the detection contact, the scent delivery device is configured to measure the resistance of the cartridge resistor. The scent delivery device may then infer the scent indication (e.g. scent ID) and/or the set of parameters for atomisation by comparison of the measured resistance with a resistance-scent indication and/or set of parameters lookup table or equivalent. The lookup table may be stored locally by a memory of the scent delivery device, or by a remote server with which the scent delivery device is configured to wireless communicate. Alternatively, the scent delivery device may be configured to communicate, preferably wirelessly, with a remote device (such as a VR headset, smartphone, or other computing device), wherein the remote device is configured to infer the scent indication (e.g. scent ID) and/or the set of parameters for atomisation based on a comparison of the measured resistance received from the scent delivery device with a resistance-scent indication and/or set of parameters lookup table or equivalent. In response, the scent delivery device may then receive control signals from the remote device, based on the identified scent ID and/or the set of parameters for atomisation.

In another example, the scent delivery device may comprise a detection means, wherein the detection means comprises a plurality of detection electrical contacts, such as but not limited to a plurality of pogo pins. In such examples, the scent cartridge may also comprise a plurality of electrical contacts, such as a plurality of pogo pins. Each electrical contact on the cartridge may represent one bit in a scent ID. For example, cartridges comprising 8 electrical contacts may comprise an eight-digit binary scent ID. The electrical contacts of the scent cartridge may be configured to electrically contact the plurality of detection contacts in the scent delivery device when the scent cartridge is installed within the scent delivery device. Upon electrical connection between the cartridge contact and the detection contact, the scent delivery device is configured to identify the scent cartridge by reading each of the pins which together provide a binary scent ID (e.g. “10011011”). The set of parameters for atomisation may be inferred by comparison of the binary scent ID with a lookup table or equivalent. Alternatively, each scent cartridge may have a specific shape, whereby when inserted into the scent delivery device, only a portion of the plurality of detection electrical contacts form an electrical contact with the cartridge. The scent ID and/or set of parameters for atomisation may be inferred by comparison with a lookup table based on the number of and/or arrangement of the portion of detection contacts which form an electrical connection with the scent cartridge.

The scent delivery device may further comprise at least one vent configured to draw air into the housing. This may be advantageous to clear lingering scents from the air surrounding the device. Alternatively, or in addition, the vent may be in fluid communication with the outlet such that the air drawn in through the vent may be configured to be expelled through the outlet, for example driven by a fan, to improve dispersion of atomised scented media released through the outlet.

In another aspect of the invention, there is provided a scent delivery device for treatment or monitoring of anosmia. The scent delivery device comprises a housing comprising an outlet, a cartridge configured to hold a scented media arranged within the housing, and an atomiser element configured to couple to the cartridge. The atomiser element is configured to atomise scented media held by the cartridge, wherein the cartridge is in fluid communication with the outlet such that atomised scented media is configured to leave the housing via the outlet. The scent delivery device further comprises a sensor configured to determine a concentration of the scented media released from the cartridge during atomisation.

This device may be advantageous to treat and/or monitor anosmia, characterised by a loss or reduction of sense of smell, as a user is able to test and track their ability to detect smells of known concentrations of the scented media, as measured by the sensor.

The sensor may be configured to detect the concentration of the scented media released from the replaceable cartridge during atomisation, for example but not limited to using a gas sensor or flowmeter. Alternatively, or in addition, the sensor may be configured to determine a concentration of the scented media released from the cartridge during atomisation by measuring the atomisation time for the cartridge, wherein atomisation time can be correlated to a concentration of the scented media released by atomisation based on known properties of the scented media.

The device may comprise a plurality of cartridges such that the device is configured to hold a plurality of scented media. This may be advantageous to treat and/or monitor anosmia as a user is able to test and track their ability to detect and distinguish between a plurality of smells at known concentrations of different scented media.

Alternatively, or in addition, the device may be configured to atomise and release more than one scent at the same time. This may be advantageous to render a complex scent. The sensor may be arranged within the housing such that the sensor is configured to determine concentration of the scented media released at the outlet. This may be advantageous to detect the concentration of the scented media at the point of release from the housing, to most accurately measure the concentration as delivered to a user.

The scent delivery device may further comprise a processor configured to control operation of the atomiser element, wherein the processor is configured to control the atomiser element to vary the concentration of the scented media released from the cartridge during atomisation across a time window. For example, the processor may be configured to control the atomiser element to increase the concentration of the scented media released from the cartridge during atomisation across a time window. By increasing the concentration over a time window, this may be advantageous for a user to test and track the lowest concentration of a scented media that they are able to detect.

The scent delivery device may further comprise a wireless communications interface configured to receive control signals from a remote device, wherein each atomiser element is configured to be controlled based on control signals received from a remote device via the wireless communications interface. Example remote devices may include, but are not limited to, a user computing device, such as a smart phone, or any other remote computing device.

The atomiser element may comprise a piezoelectric element. In such examples, the processor may be configured to control the piezoelectric element to vary the concentration of the scented media released from the cartridge during atomisation by controlling at least one of (i) the duration of vibration, and (ii) the frequency of vibration of the piezoelectric element.

The scent delivery device may further comprise a communications interface configured to send indications of the concentration sensed by the sensor to a remote device. This may be advantageous to share the sensed concentrations of the scented media with an external device to track and/or store data relating to a user's ability to detect smells. For example, the indications of the concentration sensed by the sensor may be sent to a user's personal device, such as a smartphone or laptop, for example via an app. Alternatively, or in addition, the indications of the concentration sensed by the sensor may be sent to a health professional.

The scent delivery device may further comprise a user input interface configured to receive user input relating to the user's perception of smell of the atomised scented media. The user input interface may comprise a touchscreen. Alternatively, or in addition, the user input interface may comprise at least one button. This may be advantageous to enable the device to capture data relating to the user's perception of smell of the atomised scented media which can be correlated to the concentration of scented media sensed by the sensor. Alternatively, or in addition, in embodiments comprising a plurality of cartridges comprising a plurality of different scented media, the user input interface may be advantageous to capture data relating to the user's perception of smell of the atomised scented media which can be correlated to the type of scented media released by control on the atomiser element.

The scent delivery device may further comprise at least one cleaning means, wherein each cleaning means is configured to clean the at least one atomiser element. The cleaning means may comprise at least one cleaning cavity configured to hold cleaning fluid, wherein the cleaning cavity is configured to release cleaning fluid to clean at least one atomiser element. This may be advantageous to clean away any residual scented media on the piezoelectric element, for example from a previous use or previous cartridge.

The at least one cartridge may be configured to be replaceable. Optionally, the scent delivery device may further comprise at least one cartridge sensor, each cartridge sensor being configured to detect an indication of the amount of scented media within at least one replaceable cartridge arranged within the housing. This may be advantageous to alert a user when a cartridge needs to be replaced or refilled, or when a cartridge will soon need to be replaced or refilled.

For example, the cartridge sensor may be configured to detect when the amount of scented media within the replaceable cartridge is below a predetermined threshold.

Alternatively, or in addition, the cartridge sensor may be the same as the first sensor, such that it is configured to detect concentration of the scented media released from the replaceable cartridge during atomisation.

Wherein each cartridge is configured to be replaceable, optionally each replaceable cartridge may comprise a scent indication, wherein the scent indication comprises a set of parameters for atomisation of the scented media. In such examples, the scent delivery device may further comprise a detection means configured to detect the scent indication, and a processor configured to control the atomiser element based on the detected scented indication. For example, the scent indication may comprise a scent label, such as a QR code, or other encoded indication associated with the scented media contained within the cartridge, such as a radio-frequency identification (RFID) or near-field communication (NFC) antenna. The set of parameters for atomisation may include a delivery pattern for atomisation. For example, for a scented media configured to be atomised by a piezoelectric element, the set of parameters may include at least one of (i) an optimum duration, or range of duration, of vibration of a piezoelectric element, and (ii) an optimum frequency, or range of frequencies, of vibration of a piezoelectric element, suitable for atomising the particular scented media. In this example, the scent delivery device may comprise a detection means configured to scan the QR code to retrieve the set of parameters, and a processor configured to control the piezoelectric element based on the set of parameters retrieved by the detection means.

The scent delivery device may further comprise at least one vent configured to draw air into the housing. This may be advantageous to clear lingering scents from the air surrounding the device. Alternatively, or in addition, the vent may be in fluid communication with the outlet such that the air drawn in through the vent may be configured to be expelled through the outlet, for example driven by a fan, to improve dispersion of atomised scented media released through the outlet.

### DRAWINGS

Embodiments of the disclosure will now be described, by way of example only, with reference to the accompanying drawings, in which:

FIG. 1A shows a top view of an example wearable scent delivery device.

FIG. 1B shows a front view of the example scent delivery device of FIG. 1A.

FIG. 1C shows a bottom view of the example scent delivery device of FIGS. 1A and 1B.

FIG. 1D shows a side view of the example scent delivery device of FIGS. 1A to 1D.

FIG. 2 shows an example wearable scent delivery device, such as the device of FIGS. 1A to 1D, in use as worn by a user.

FIG. 3 shows a schematic block diagram of the internal components of an example scent delivery device, such as the device of FIGS. 1A to 2.

FIG. 4 shows a schematic virtual scent map to illustrate how a scent delivery device, such as any device of FIGS. 1A to 3, may be actuate based on location of the device.

FIGS. 5A-F each show a different example scent cartridge configuration for use with a scent delivery device, such as the scent delivery device of FIGS. 1A to 3.

FIG. 6A shows an example scent cartridge, such as any of the cartridges FIG. 5A to FIG. 5D, being coupled to a piezoelectric element, wherein the piezoelectric element is arranged within a lid configured to couple to the cartridge.

FIG. 6b shows the scent cartridge and lid of FIG. 6a in an assembled state.

FIG. 7 shows another example scent cartridge being coupled to a piezoelectric element, wherein the piezoelectric element is arranged within a lid configured to couple to the cartridge. The lid comprising an element configured to extend through a membrane of the scent cartridge.

FIG. 8A shows a cross section of an example replaceable cartridge, such as any of the cartridges FIG. 5A to FIG. 5D, being coupled to a scent delivery device by a bump fit.

FIG. 8B shows the coupled configuration of the replaceable cartridge and scent delivery device of FIG. 8A.

FIG. 9A shows a cross section of an example replaceable cartridge, such as any of the cartridges FIG. 5A to FIG. 5D, being coupled to a scent delivery device by a bayonet fit.

FIG. 9B shows the coupled configuration of the replaceable cartridge and scent delivery device of FIG. 9A.

FIG. 10A shows a cross section of an example replaceable cartridge, such as any of the cartridges FIG. 5A to FIG. 5D, being coupled to a scent delivery device by a biased push fit.

FIG. 10B shows the coupled configuration of the replaceable cartridge and scent delivery device of FIG. 10A.

FIG. 11A shows an exploded, isometric view of an example replaceable cartridge, such as any of the cartridges FIG. 5A to FIG. 5D, configured to be coupled to a scent delivery device by a snap fit.

FIG. 11B shows an exploded side view of the replaceable cartridge and portion of the scent delivery device of FIG. 11A.

FIG. 11C shows a cross-section view of the replaceable cartridge and portion of the scent delivery device of FIGS. 11A and 11B in the assembled configuration.

FIG. 12A shows a perspective view of another example replaceable cartridge, configured to be coupled to a scent delivery device, such as the device of FIG. 1A to FIG. 2.

FIG. 12B shows a cross-section view of the replaceable cartridge of FIG. 12A.

FIG. 13A shows a cross section of a schematic of electrical contacts within a scent delivery device, configured to receive a scent cartridge.

FIG. 13B shows a cross section of the schematic of FIG. 13A, wherein electrical contacts of a scent cartridge are coupled to electrical contacts of the scent delivery device.

### SPECIFIC DESCRIPTION

Embodiments of the claims relate to scent delivery devices and replaceable scent cartridge for use therewith.

It will be appreciated from the discussion above that the embodiments shown in the Figures are merely exemplary, and include features which may be generalised, removed or replaced as described herein and as set out in the claims.

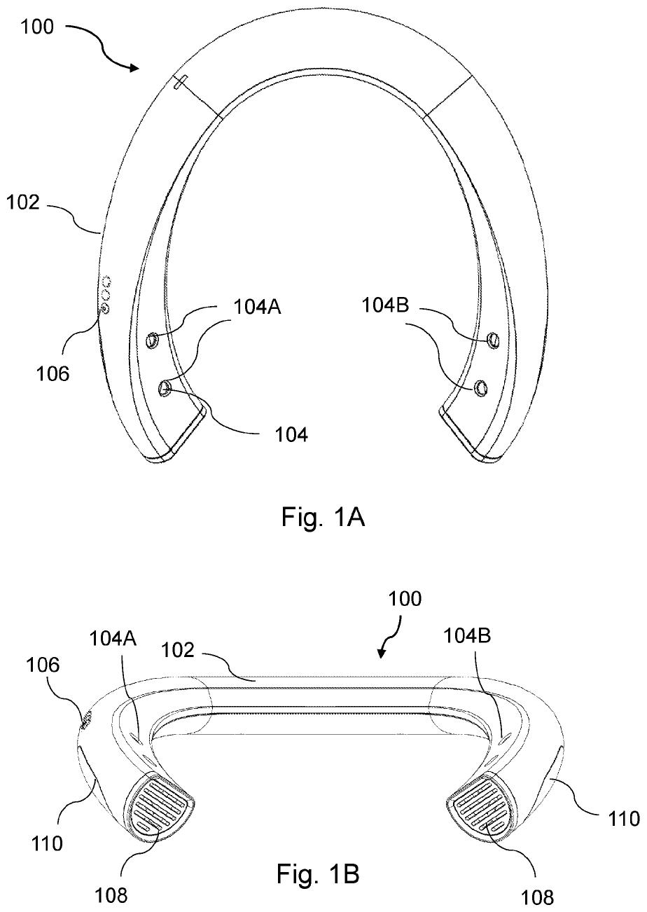

FIGS. 1A-1D show an example wearable scent delivery device 100 of the present invention. The scent delivery device 100 comprises an arch shaped housing 102. The housing 102 further comprises two sets of outlets, 104A and 104B, wherein the sets are arranged at opposing ends of the arched shaped housing 102. Each set of outlets, 104A and 104B, is arranged on an upper surface of the housing 102. This may be advantageous to direct the outlets towards the user's head in use, and avoid interference with clothing. In this example, each set of outlets comprises two outlets 104.

The housing 102 further comprises a set of control buttons 106. In this example, the control buttons 106 are arranged along one side of the arched housing 102. The control buttons 106 are preferably arranged on an outwards facing surface of the housing 102 to facilitate easy access by the user in use.

As shown in FIG. 1B, the arched shaped housing 102 has a substantially tubular cross-section. A vent 108 is arranged at each terminal cross-sectional end of the arch shaped housing 102.

The arch shaped housing 102 also comprises a cartridge door 110 at each end. In this example, the cartridge door is arranged on the opposite surface of the housing 102 to the outlets 104.

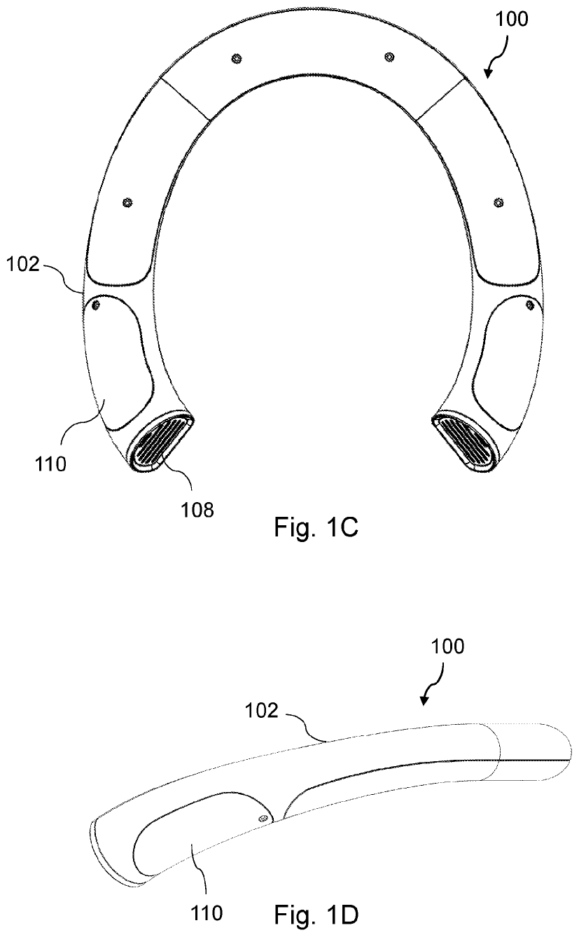

The arch shaped housing 102 is configured to be worn around the neck of a user, as shown in FIG. 2. The skilled person will understand that the arch shaped housing 102 is not required to completely surround or enclose the neck of a user.

The housing 102 shown in FIG. 2 is made of a rigid material, such as a rigid polymer.

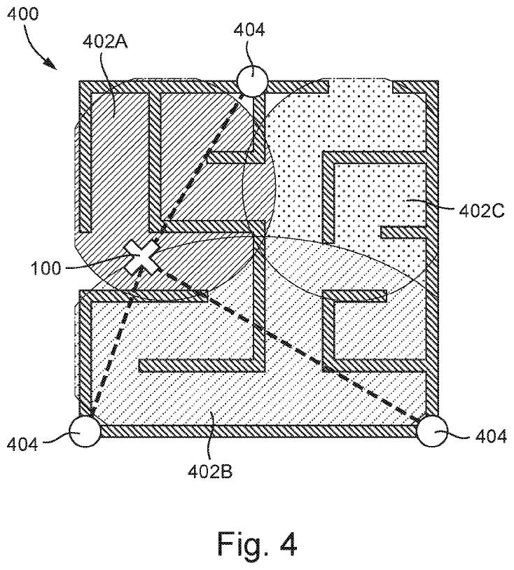

The housing 102 is configured to hold a plurality of scent cartridges (not shown). A selection of example scent cartridges are shown in FIGS. 5A to 7.

The cartridges are configured to be accessed through the cartridge door 110. This may allow the scent cartridges to be replaced and/or interchanged.

Atomised scented media from the scent cartridges is configured to be released from the device 100 via the outlets 104.

The control buttons 106 are configured to control operation of the device 100. Example control functionality may include, but is not limited to, powering the device 100 on and/or off, controlling the intensity/concentration of scented media released (e.g., increasing or decreasing intensity/concentration), and pairing and/or unpairing the device 100 to a remote device by way of wireless communication.

The vent 108 is configured for air intake into the housing 102. Optionally, a fan (not shown) may be arranged behind the vent 108 within the housing 102.

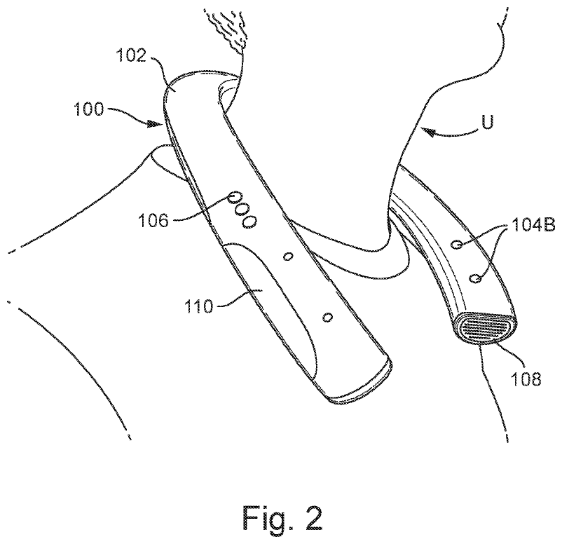

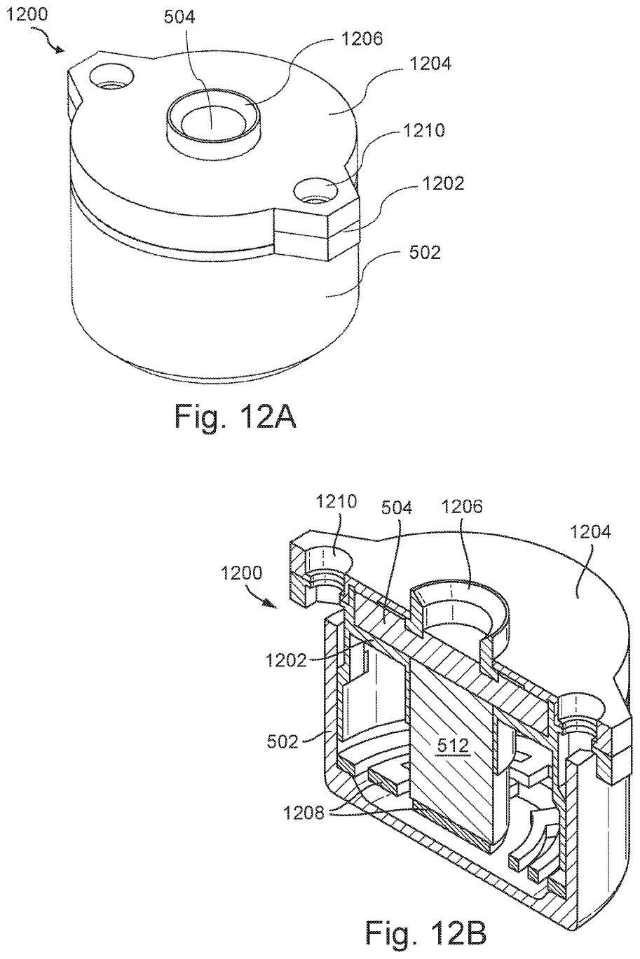

An example block diagram of the internal components of a scent delivery device, such as the device 100 of FIGS. 1A to 2, is shown in FIG. 3. Within the housing 102, the device 100 comprises a controller 302, coupled to a plurality of atomiser elements 306. In this example, the atomiser elements 306 each comprise a piezoelectric element. In use, each atomiser element 306 is coupled to a scent cartridge 308, however the skilled person will understand that the scent cartridges 308 may be configured to be removed from and/or replaced in the housing 102. The skilled person will also understand that, in some embodiments, the atomiser elements 306 may be integral to the scent cartridges 308, for example as shown in FIG. 12B, wherein the scent cartridges 308 (including their respective atomiser elements 306) may be configured to be removed from and/or replaced in the housing 102 The atomiser elements 306 are each configured to atomise scented media within the respective scent cartridge 308. The processor 302 is configured to control actuation of each atomiser element 306 independently to control atomisation. The scent delivery device further comprises a power source (not shown), for example wherein the power source may be a rechargeable battery. The power source is configured to power the internal components of the scent delivery device, including, but not limited to the controller 302 and the plurality of atomiser elements 306.

The processor 302 is also coupled to a fan 312, wherein the processor 302 is also configured to actuate the fan 312. The fan 312 is configured to disperse atomised scented media released by the scent cartridges 308. Alternatively, or in addition, the fan 312 may also be configured to draw air into the housing 102 and filter the air in order to remove lingering scents outside of the housing 102.

The device 100 further comprises a wireless communication means 304, coupled to the processor 302. The wireless communication means 304 is configured to receive control signals from a remote device. The processor 304 is then configured to actuate the plurality of atomiser elements 306 in accordance with the received control signals or instructions.

Optionally, the wireless communication means 304 may further comprise a means for determining location of the device 100. For example, the wireless communication means 304 may comprise a GPS receiver, Bluetooth receiver, or any other suitable means. In such example, the processor 304 may be configured to actuate the plurality of atomiser elements 306 based on the location of the device 100.

Optionally, the device 100 further comprises an inertial measurement unit (IMU) 310, wherein each atomiser element 306 is configured to be controlled by the processor 302 based on an indication determined by the IMU 310. For example, the IMU 310 may comprise at least one of an accelerometer, gyroscope, and/or magnetometer.

Returning to the example shown in FIGS. 1A-2, in use a user U wears the housing 102 around their neck such that the mid portion of the arched housing 102 is adjacent to the back of the user's neck, as shown in FIG. 2.

In use, the first set of outlets 104A is arranged on the opposite side of sagittal plane of the user U to the second set of outlets 104B. In this example, all outlets 104 are configured to be positioned forwards of the frontal (or coronal) plane of a user in use, wherein forwards of the frontal plane corresponds to the anterior direction. This may be advantageous to effectively deliver the released scents in the proximity of the user U's nose.

The processor 306 controls the operation of the plurality of atomiser elements 306 independently.

Optionally, each scent cartridge 308 comprises a scent indication, wherein the scent indication comprises a set of parameters for atomisation of the scented media contained within the respective cartridge 308. For example, the scent indication may comprise a scent label, such as a QR code, or other encoded indication, such as an RFID or NFC antenna, associated with the scented media contained within the cartridge. A detection means within the device (not shown) detects the scent indication to retrieve the set of parameters for atomisation, the set of parameters may vary for different scents and/or types of scented media For example, for a scented media configured to be atomised by a piezoelectric element, the set of parameters may include at least one of (i) an optimum duration, or range of duration, of vibration of a piezoelectric element, and (ii) an optimum frequency, or range of frequencies, of vibration of a piezoelectric element, suitable for atomising the particular scented media. These parameters may be different for different scents, for example based on their chemical properties. The processor 306 is then configured to control the operation of each atomiser element 306 according to the set of parameters for the associated scent cartridge 308.

When actuated, an atomiser element 306 atomises scented media contained within one of the scent cartridges 308. Preferably, the atomiser elements 306 are microporous piezoelectric elements. The atomised scented media then diffuses out of the cartridge 308 through the microporous piezoelectric element, and out of the housing 102 via one of the outlets 104. Diffusion of the scented media may be aided by operation of the fan 312 within the housing. The fan 312 may draw air into the housing 102 through the vent 108, creating an airflow which carries the scented media out through one of the outlets 104. Once released, the user U is then able to smell the atomised scented media.

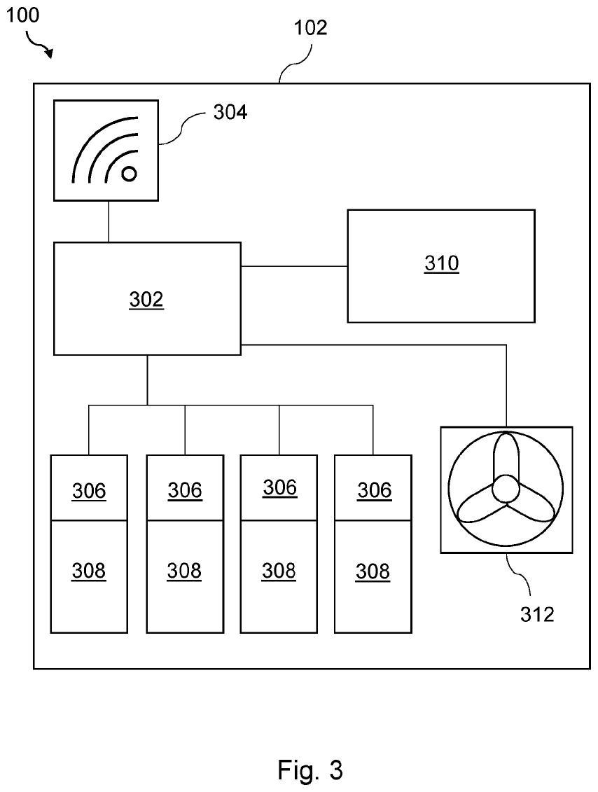

In some examples, the processor 302 may be configured to control the operation of the atomiser elements 306 based on an indication of location of the user, wherein the location of the user is sensed by a location sensor, such as a wireless location receiver, in the device 100. An example use case of location-dependent actuation is illustrated in FIG. 4 which shows an example virtual map 400. When the device 100 is sensed to be within a first area 402A, the processor 302 is configured to actuate at least a portion of the atomiser elements 306 to atomise and release scented media from at least one scent cartridge 308 pertaining to a first smell. Similarly, when the device 100 is sensed to be within a second area 402B, the processor 302 is configured to actuate a second portion of the atomiser elements 306 to atomise and release scented media from at least one scent cartridge 308 pertaining to a second smell, wherein the first and second smells are different, and so on. Whilst the example shown in FIG. 4 comprises three different areas, 402A, 402B, and 402C, pertaining to three different smells, the skilled person will understand that this may apply to any number of areas, associated with any number of scents. The different areas may be defined by geofencing the virtual map 400.

In this example, the location is sensed by the device 100 based on wireless communication via the wireless communication means 304 with a plurality of location receivers 404 distributed throughout the area 400. The location of the device 100 may then be determined based on triangulation methods, for example to determine 3D spatial coordinates (x, y, z), or any other suitable technique. In other examples, location of the device 100 may be sensed by way of GPS or other satellite-based location technique.

This may have applications for use in immersive art and science exhibitions, for example wherein smells are released based on the exhibition space the user U is in.

Alternatively, this functionality may be used for emergency services training, for example for training exercises relating to inspecting an area including a virtual gas leak, or similar. As an example, the device 100 may be configured to increase the intensity of scent released by the device 100 as the sensed location of the device 100 approaches the virtual “source” of the scent, wherein the virtual source is marked on the virtual map 400. Increasing the intensity of the scent may comprise at least one of: (i) increasing the frequency of actuation of a piezoelectric element coupled to the relevant scent cartridge, (ii) increasing the duration of actuation of the relevant piezoelectric element, or (iii) actuating more atomiser elements coupled to scent cartridges of the relevant smell.

Alternatively, or in addition, in some examples, the processor 302 may be configured to control the operation of the atomiser elements 306 based on an indication of movement of the user U wearing the device 100, sensed by the device 100's IMU 310.

This may be advantageous to provide the user with a sense of directional smell. For example, the IMU 310 may be configured to sense the user's angular positioning relative to a virtual source of the scent according to a virtual scent map. For example, if the IMU 310 senses that the user is facing away from the virtual source of the scent, the processor 302 may be configured to actuate the atomiser element(s) 306 located on a side of the device 100 closest to the virtual source and corresponding to the relevant scent. For example, if the IMU 310 senses that the virtual source is located to the left of the device 100, the processor 302 will actuate the atomiser element(s) 306 coupled to the outlets 104B located on the left-hand side of the device 100 corresponding to the relevant scent. As such, the scent is released from the left-hand side outlets 104B only, and the user U will perceive the source of the scent to be to the left. As the IMU 310 senses the user turning towards the source of the scent, the processor 302 may increase the intensity of the scent released. By contrast, if the IMU 310 senses the user turning away from the virtual source of the scent, the processor 302 may reduce the intensity of the scent released.

Whilst the housing 102 shown in FIG. 1A-2 is made of a rigid material, such as a rigid polymer, the skilled person will understand that in other examples at least a portion of the housing may be flexible or adjustable. For example, in some examples, the mid-portion of the arch shaped housing 102 may comprise an extendable portion, configured to extend and/or compress the length of the arch shaped housing. The extendable portion is preferably configured to be arranged to be adjacent to the back of the neck of a user in use. For example, the extendable portion may comprise a concertinaed portion, configured to be extended and/or compressed. Alternatively, or in addition, the end portions of the arched housing 102 comprising the outlets 104 may be configured to be repositioned relative to the rest of the housing 102, for example the end portions comprising the outlets 104 may be configured to be bendable.

FIG. 5A to E show a range of example scent cartridges for use in a scent delivery device, such as the scent delivery device 100 described herein.

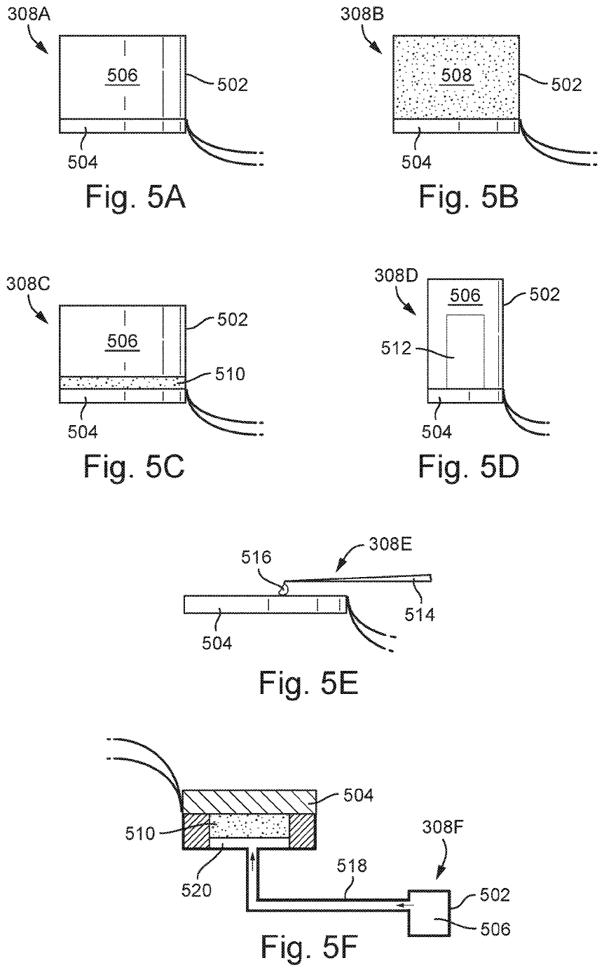

FIG. 5A shows a first scent cartridge 308A comprising a container 502. In this example, the container 502 is configured to hold scented liquid 506. The open end of the container 502 is coupled to a piezoelectric element 504. The piezoelectric element 504 is configured to atomise the scented liquid 506. The piezoelectric element 504 is microporous such that atomised liquid leaves the cartridge 308A via the piezoelectric element 504 micropores. An example coupling between a container 502 and piezoelectric element 504 is shown in FIGS. 6A-6B.

FIG. 5B shows another scent cartridge 308B comprising a container 502. In this example, the container 502A is configured to hold porous material 508 soaked in scented liquid. The open end of the container 502 is coupled to a piezoelectric element 504 such that the porous material 508 is configured to contact the piezoelectric element 504. The porous material 508 is configured to transport scented liquid to the piezoelectric element 504 by capillary action. An example coupling between a container 502 and piezoelectric element 504 is shown in FIGS. 6A-6B. Although the piezoelectric element 504 is arranged below the container 502 in FIG. 5B, the skilled person will understand that in other examples, the piezoelectric element 504 may be arranged above the container 502, wherein the scented media is supplied to the piezoelectric element 504 by capillary action.

FIG. 5C shows another scent cartridge 308C. This scent cartridge 308C comprises a container 502 configured to hold scented liquid 506. The scent cartridge 308C further comprises a layer 510 of porous material arranged between the liquid 506 and a piezoelectric element 504. The layer 510 of porous material forms a porous membrane between the liquid 506 and the piezoelectric element 504. This may be advantageous to prevent or reduce liquid blocking the pores within the piezoelectric element 504.

FIG. 5D shows another scent cartridge 308D. This scent cartridge 308D comprises a container 502 configured to hold scented liquid 506. The scent cartridge 308C further comprises a wick element 512 of porous material arranged between the liquid 506 and a piezoelectric element 504, such that the wick element 512 contacts the piezoelectric element 504. Scented liquid 506 is configured to be supplied to the piezoelectric element 504 via the wick element 512, in a similar manner to a marker pen. The wick element 512 is preferably arranged approximately central to the container 502. The liquid 506 is sealed from the piezoelectric element 504 such that is does not directly contact the piezoelectric element 504, except via the wick element 512. In the example shown, the wick element 512 does not extend the entire length of the container, however the skilled person will understand that in other examples, the wick element 512 may extend substantially the entire length of the container 502. The wick element 512 also preferably protrudes out of the container 502, wherein the exposed end of the wick element 512 is configured to contact the piezoelectric element 504, akin to the nib of a marker pen. In some examples, the wick element 512 may be made of a fibrous material, such as fibrous polyester.

Although the piezoelectric element 504 is arranged below the container 502 in FIG. 5D, the skilled person will understand that in other examples, the piezoelectric element 504 may be arranged above the container 502, wherein the scented media is supplied to the piezoelectric element 504 by capillary action.

In the embodiment shown in FIG. 5E, scented liquid drops 516 are dispensed by a dispensing means 514 onto the piezoelectric element 504 to be atomised. The piezoelectric element 504 is arranged directly below the dispensing means 516, such that the liquid drops 516 contact the piezoelectric element 504 under gravity. The dispensing means 514 may comprise small pump, piezo pump, pipette, or any other suitable means. In the embodiment shown in FIG. 5F, a container 502 configured to hold scented liquid 506 is displaced from the piezoelectric element 504, such that they are not in contact. Instead, the scent contained 502 is coupled to the piezoelectric element 504 by a tube 518. This may be advantageous to allow the scent cartridge(s) 306F to be replaced and interchanged within a scent delivery device by a user at a location separate from the piezoelectric elements 504. This may improve the accessibility of the cartridges 306F within a scent delivery device. Locating the container 502 at a location separate from the piezoelectric element 504 may also allow the container 502 to have a larger volume, for example as the container 502 is subject to fewer size constraints. This may be advantageous to allow the container 502 to hold a greater capacity of scented liquid.

The scented liquid may be configured to be pumped through the tube 518 from the container 502. Alternatively, the scented liquid may be configured to be drawn through the tube 518 by capillary action, for example wherein the tube is sized accordingly.

At the end of the tube 518 adjacent to the piezoelectric element 504, a wick element of porous material 510 arranged within a chamber 520 separates the tube 518 and the piezoelectric element 510. The scented liquid is configured to be absorbed by the porous material 510 to contact the piezoelectric element 504 for atomisation. The wick element of porous material 510 acts as a porous membrane to control delivery of the scented liquid to the piezoelectric element 504.

FIG. 6A shows an example embodiment of a scent cartridge 308, such as any of the scent cartridges 306A to 306D shown in FIGS. 5A to 5D respectively, being coupled to a piezoelectric element 504. As shown, the piezoelectric element 504 is arranged in a lid portion 602, wherein the lid portion 602 is configured to be coupled to an open end of the container 502. The piezoelectric element 504 is arranged on an inner surface of the lid such that the scented media contained within the cartridge 308 is configured to contact the piezoelectric element 504 when the lid 602 is coupled to the cartridge 308. The piezoelectric element 504 is microporous, comprising pores extending to the outer surface of the lid portion 602. As such, scented media atomised by the piezoelectric element 504 is configured to escape from the cartridge 308 via the pores in the piezoelectric element 504. This is illustrated in FIG. 6B which shows the scent cartridge 308 and lid portion 602 of FIG. 6A in the coupled configuration.

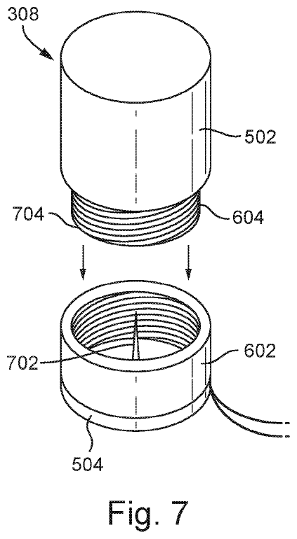

The coupling between the cartridge 308 and the lid portion 602 is reversible. This may be advantageous to allow the same lid portion 602 and piezoelectric element 504 to be used with a plurality of cartridges 306 as the piezoelectric element 504 has a longer usable life compared to a replaceable cartridge 308 which is configured to contain a finite source of scented media. In the example shown in FIG. 6A, the neck of the container 502 comprises a screw thread 604 and is configured to couple to the lid portion 602 by screw closure, wherein the inner circumference of the lid portion 516 comprises a complementary screw thread. However, the skilled person will understand that screw closure is only one example of many possible reversible couplings, including but not limited to snap fits, push fits, bayonet closures, and any other suitable means. A non-exhaustive selection of suitable reversible coupling mechanisms is shown in FIGS. 8A to 10B.

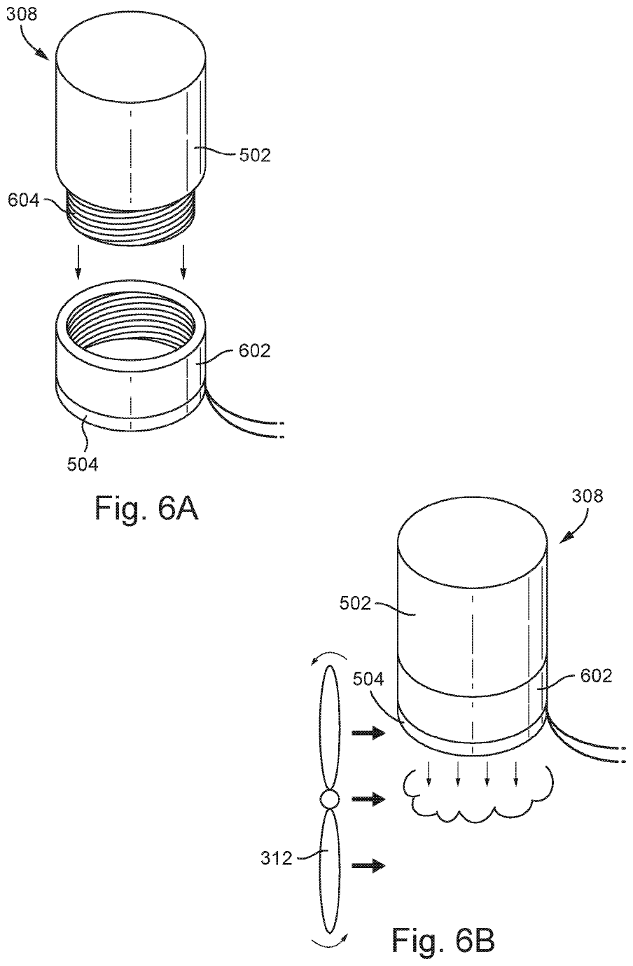

FIG. 7 shows an example embodiment of a resealable scent cartridge 308 for use in a scent delivery device, such as the device 100 of any of FIGS. 1 to 3, being coupled to a piezoelectric element 504. The resealable cartridge 308 comprises a container 502 configured to hold scented liquid, and an impermeable membrane (not shown) comprising at least one resealable aperture. The impermeable membrane is arranged across the opening 704 of the container 502. The impermeable membrane is configured to seal the resealable aperture in a closed configuration, and unseal the resealable aperture in an open configuration. The impermeable membrane is also configured to seal the container 502 in the closed configuration, such that scented liquid is configured to be sealed within the container 502 in the closed configuration, and scented liquid is configured to leave the container 502 via the resealable aperture in the open configuration.

As with FIG. 6A, the piezoelectric element 504 is arranged in a lid portion 602, wherein the lid portion 602 is configured to be coupled to the scent cartridge 308. The piezoelectric element 504 is arranged on an inner surface of the lid 602 such that the resealable aperture is configured to be adjacent to the piezoelectric element 504 when the lid 602 is coupled to the cartridge 308. The piezoelectric element 504 is microporous with pores extended to the outer surface of the lid portion 602. As such, scented liquid atomised by the piezoelectric element 504 is configured to escape from the cartridge 308 via the pores in the piezoelectric element 504. In this example, the cartridge 308 comprises a screw thread 604 and is configured to couple to the lid portion 602 by screw closure. However, the skilled person will understand that screw closure is only one example of many possible reversible couplings, including but not limited to snap fits, push fits, bayonet closures, and any other suitable means. A non-exhaustive selection of suitable reversible coupling mechanisms is shown in FIGS. 8A to 10B.

The lid portion 602 further comprises protruding structure 702 coupled to the inner surface of the lid, opposing the impermeable membrane of the cartridge 604. When the lid 602 is coupled to the cartridge 308, the protruding structure 702 is configured to extend through the resealable aperture. As such, the protruding structure 702 is configured to bias the impermeable membrane into the open configuration by deforming the impermeable membrane. In the example shown, the protruding structure 702 is a hollow tube, wherein scented liquid is configured to be drawn into the hollow tube, for example by capillary action, and transferred to the surface of the piezoelectric element 504 for atomisation via the tube.

Optionally, the protruding structure 702 may comprise a sensor configured to detect an indication of the amount of scented media within the cartridge 308, wherein the sensor is configured to extend through the resealable aperture into the container 502.

The skilled person will understand that, in some examples, the lid portion 602 shown in FIGS. 6A to 7 may be integrated within an internal surface of the device housing 102, for example as shown in FIGS. 8A to 10B.

FIGS. 8A and 8B show cross section of an example replaceable cartridge 308, such as any of the cartridges FIG. 5A to FIG. 5D, being coupled to a scent delivery device, such as the scent delivery device 100, by a bump fit. A portion of the device housing 102 is shown comprising an outlet 104. An atomiser element, in this example a piezoelectric element 504, is arranged within the outlet 104. The inner surface of the device housing 102 comprises a lid portion 602, wherein the lid portion 602 is configured to receive and couple to a replaceable cartridge 308. The inner surface of the lid portion 602 comprises at least one engagement structure 802. In the example shown, the engagement structures 802 form a continuous flange around the inner circumference of the lid portion 602, however in other examples the skilled person will understand that the inner surface of the lid portion 602 may instead comprise a plurality of discrete engagement structures 102.

The cartridge 308 comprises a container 502. In the example shown, the container 502 comprises a porous material 510 soaked in scented liquid. In this example, the porous material 510 is configured to protrude out of the container 502, akin to a wick element. The outer surface of the container 502 comprises at least one complementary engagement structure 804, wherein the complementary engagement structure 804 is configured to have a complementary shape to the engagement structure 802. In this example, the complementary engagement structure 804 comprises a circumferential groove.

As shown in FIG. 8B, the complementary engagement structure 804 is configured to engage with the engagement structures 802 of the housing 102 in order to couple the cartridge 308 to the housing 102. When coupled, the porous material 510 is configured to contact the piezoelectric element 504.

The complementary engagement structure 804 and housing engagement structure 802 may be engaged by a snap fit. Preferably, cartridge 308 is configured to be at least partially resiliently deformable to accommodate the snap fit. The cartridge 308 may then be deformed to disengage the engagement structures 804 and 802 in order to remove and/or replace the cartridge 308.

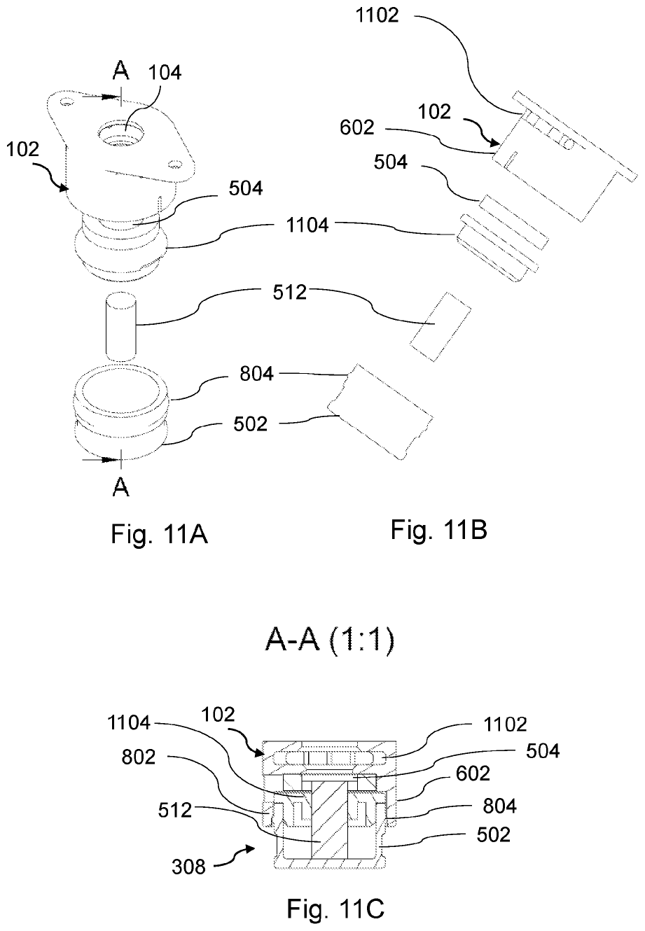

An alternative embodiment of cartridge 308 is shown in FIGS. 11A to 11C. This shows a portion of the device housing 102 comprising an outlet 104. An atomiser element, in this example a piezoelectric element 504, is arranged within the outlet 104. The inner surface of the device housing 102 comprises a lid portion 602, wherein the lid portion 602 is configured to receive and couple to a replaceable cartridge 308. The lid portion 602 comprises an aperture 1102, configured to be arranged between the piezoelectric element 504 and the outlet 104, wherein the aperture 1102 is configured to allow the air from a fan installed within the scent delivery device, such as device 100, to flow between the piezoelectric element 504 and the outlet 104, for example to improve the flow of atomised particles.

The inner surface of the lid portion 602 also comprises at least one engagement structure 802. In the example shown, the engagement structure 802 is a continuous circumferential groove around the inner circumference of the lid portion 602.

The cartridge 308 comprises a container 502 and a container lid 1104. In the example shown, the container 502 comprises a porous material soaked in scented liquid which is arranged to form a wick 512. The wick 512 is configured to protrude out of the container 502 and container lid 1104, wherein the container lid 1104 comprises a central aperture. The outer surface of the container 502 comprises at least one complementary engagement structure 804, wherein the complementary engagement structure 804 is configured to have a complementary shape to the engagement structure 802. In this example, the complementary engagement structure 804 comprises a circumferential flange.

As shown in FIG. 11C, the complementary engagement structure 804 is configured to engage with the engagement structures 802 of the housing 102 in order to couple the cartridge 308 to the housing 102. When coupled, the wick 512 is configured to contact the piezoelectric element 504.

The complementary engagement structure 804 and housing engagement structure 802 may be engaged by a snap fit. Preferably, cartridge 308 is configured to be at least partially resiliently deformable to accommodate the snap fit. The cartridge 308 may then be deformed to disengage the engagement structures 804 and 802 in order to remove and/or replace the cartridge 308.

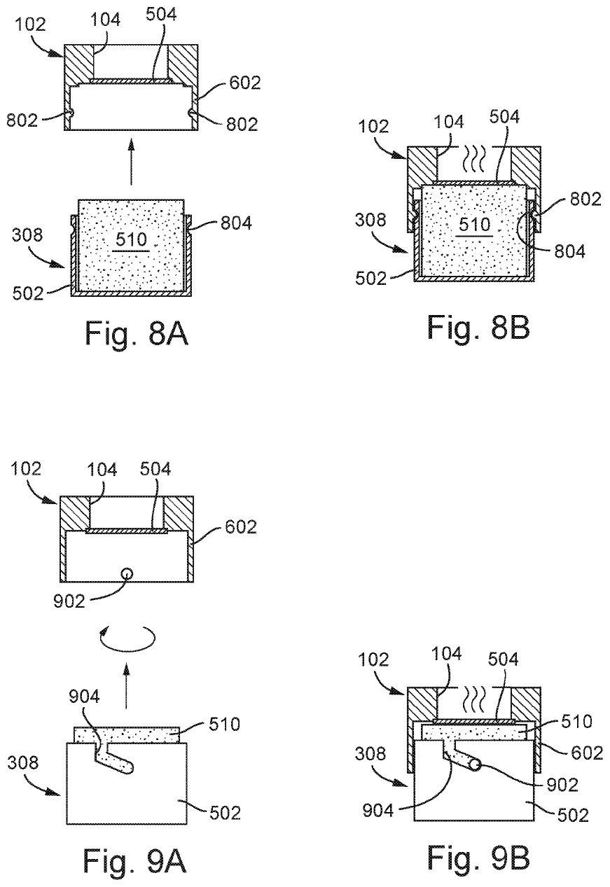

FIGS. 9A and 9B shows cross section of an example replaceable cartridge, such as any of the cartridges FIG. 5A to FIG. 5D, being coupled to a scent delivery device, such as the scent delivery device 100, by a bayonet fit. A portion of the device housing 102 is shown comprising an outlet 104. An atomiser element, in this example a piezoelectric element 504, is arranged within the outlet 104. The inner surface of the device housing 102 comprises a lid portion 602, wherein the lid portion 602 is configured to receive and couple to a replaceable cartridge 308. The inner surface of the lid portion 602 comprises a pair of engagement structures 902 arranged on opposing sides of the lid portion 602. In the example shown, each engagement structure 902 is a cylindrical protrusion.

The cartridge 308 comprises a container 502. In the example shown, the container comprises a porous material 510 soaked in scented liquid. In this example, the porous material 510 is configured to protrude out of the container 502, akin to a wick element. The outer surface of the container 502 comprises a pair of engagement structures 904 arranged on opposite sides of the container 502. In this example, each engagement structure 904 comprises a bayonet groove mount. The bayonet groove mount has a dog-leg shape. As shown in FIG. 9B, the bayonet groove mount 904 is configured to engage with the engagement protrusions 802 by applying a twisting motion relative to one another, thus coupling the cartridge 308 to the housing 102. When coupled, the porous material 510 is configured to contact the piezoelectric element 504.

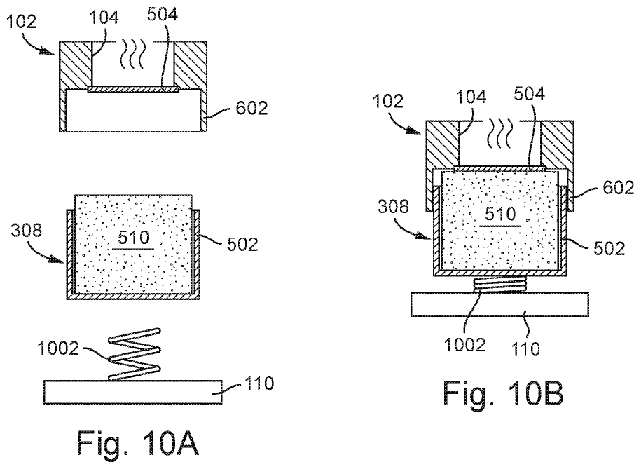

FIGS. 10A and 10B show cross section of an example replaceable cartridge, such as any of the cartridges FIG. 5A to FIG. 5D, being coupled to a scent delivery device, such as the scent delivery device 100, by a biased push fit. A portion of the device housing 102 is shown comprising an outlet 104. An atomiser element, in this example a piezoelectric element 504, is arranged within the outlet 104. The inner surface of the device housing 102 comprises a lid portion 602, wherein the lid portion 602 is configured to receive and couple to a replaceable cartridge 308.

The cartridge door 110 is arranged on the opposite side of the device housing 102 to the lid portion 602. The inner surface of the cartridge door 110 comprises a resilient biasing member 1002. In this example, the resilient biasing member 1002 is a spring.

The cartridge 308 comprises a container 502. In the example shown, the container comprises a porous material 510 soaked in scented liquid. In this example, the porous material 510 is configured to protrude out of the container 502, akin to a wick element.

As shown in FIG. 10B, the lid portion 602 is configured to receive the cartridge 308. When coupled, the porous material 510 is configured to contact the piezoelectric element 504. When the cartridge door 110 is closed, the cartridge 308 is configured to be retained in the lid portion 602 by the resilient biasing member 1002 which exerts a force onto the cartridge to urge the porous material 510 to contact the piezoelectric element 504. In this example, the spring is compressed against the surface of the container 502 opposite to the exposed porous material 510.

To remove and/or replace the cartridge 308, the cartridge door 110 is opened which disengages the resilient biasing member 1002 from the container 502 so the container can easily be removed.

Whilst the cartridge 308 depicted in FIGS. 8A to 10B most closely resemble the cartridge 308B of FIG. 5B, the skilled person will understand that any of the cartridges disclosed herein, in particular any of the cartridges disclosed in FIGS. 5A to 5D may be coupled to the device housing 102 in the manners described herein.

Furthermore, the skilled person will understand that the piezoelectric elements 506 shown in FIGS. 8A to 10B may optionally include a protruding structure, such as protruding structure 702 shown in FIG. 7, coupled to the inner surface of the lid portion 602. In such examples, the cartridge 308 may comprise an impermeable membrane across the opening of the container 502, wherein the protruding structure is configured to extend through the membrane in the coupled configuration, as described in relation to FIG. 7.

Another example scent cartridge 1200 for use with a scent delivery device is shown in FIGS. 12A and 12B. In this example, the piezoelectric element 504 is integrated within the cartridge 1200. This may be advantageous to avoid cross-contamination of scents as a result of exchanging or swapping cartridges within a scent delivery device. As such, this may avoid the need for cleaning of the piezoelectric element 504 between use with different scent cartridges.

This scent cartridge 1200 comprises a container 502 configured to hold scented liquid (not show). The scent cartridge 1200 further comprises a wick element 512 of porous material arranged between the liquid and a piezoelectric element 504, such that the wick element 512 contacts the piezoelectric element 504. The piezoelectric element 504 is supported by an insert 1202 into the container 502, wherein the insert 1202 is configured to provide a shelf for supporting the piezoelectric element at the open end of the container 502. The insert 1202 also provides an aperture to receive the wick element 512, such that the wick element 512 contact the piezoelectric element 504 through the aperture.

Providing the piezoelectric element 504 on the insert 1202 may be advantageous to avoid the need for the piezoelectric element to be bonded, adhered, or otherwise permanently fixed to the cartridge 1200. In this regard, the piezoelectric element may be easily removed from the cartridge, for example so that the cartridge can be refilled, or so that the piezoelectric element can be replaced if damaged. Whilst in this example, the insert 1202 is depicted as a separate part inserted into the container 504, the skilled person will understand that in other embodiments, the container 504 may be provided with an integral portion, equivalent to insert 1202, configured to support the piezoelectric element 504.

The piezoelectric element 504 is reversibly secured in position on the insert 1202 by a lid portion 1204. The lid portion 1204 is reversibly secured to the insert 1202 and/or container 502, for example by a mechanical fastening. In the example shown in FIGS. 12A and 12B, the lid portion 1204 and insert 1202 each comprise two apertures 1210 which are configured to receive each align so as to receive a mechanical fastener, such as a screw, bolt, or peg. The piezoelectric element 504 is therefore sealed between the lid 1204 and the insert 1202.

The lid portion 1204 is provided with a nozzle 1206, wherein the nozzle 1206 is in fluid communication with the piezoelectric element 504. As such, scented media atomised by the piezoelectric element 504 is configured to be released from the cartridge 1200 via the nozzle 1206. The nozzle 1206 has a raised profile from the surface of the lid 1204. In use, the nozzle 1206 is configured to protrude through an outlet of the scent delivery device. For example, when in use with the scent delivery device 100 of FIGS. 1A to 2, the nozzle 1206 is configured to protrude through one of the outlets 104, despite the cartridge 1200 being arranged inside the housing 102. This may be advantageous to ensure that all atomised scent particles are dispensed outside of the device, preventing atomised scent particles becoming trapped within the device housing 102. Therefore, the nozzle 1206 may improve the efficiency of scent delivery to the user, and reduce inadvertent mixing of scents when using multiple different scent cartridges within the same device housing 102.

As with the cartridge of FIG. 5D, scented liquid 506 is configured to be supplied to the piezoelectric element 504 via the wick element 512. The wick element 512 is preferably arranged approximately central to the container 502. The liquid 506 is sealed from the piezoelectric element 504 by the insert 1202 such that is does not directly contact the piezoelectric element 504, except via the wick element 512.

In the example shown, the wick element 512 does not extend the entire length of the container. However, in order to maintain constant contact between one end of the wick 512 and the piezoelectric element 504, a biasing means is provided to abut the opposite end of the wick element 512, therefore exerting a biasing force on the wick element 512 towards the piezoelectric element 504. In this example, an ortho-planar spring 1208 is provided as the biasing means within the container 502. Ortho-planar springs 1208 have been found to be particularly advantageous to provide a constant force against the wick element 512; however the skilled person will understand that other biasing means may be used, such as but not limited to, a conical spring, or any other spring.

In use, the piezoelectric element 504 is configured to be electrically coupled with contacts in the scent delivery device, for example, but not limited to, via a connection with pogo pins, or other electrical contacts. At least one electrical contact is configured to power and control operation of the piezoelectric element 504 by the scent delivery device. The cartridge 1200 may be configured to be retained with the device housing by a snap- or push-fit, or by magnetic attraction between magnets arranged on both the cartridge 1200 (not shown) and within the housing 102. However, the skilled person will understand that any other suitable methods for retaining a cartridge within a device may be used.

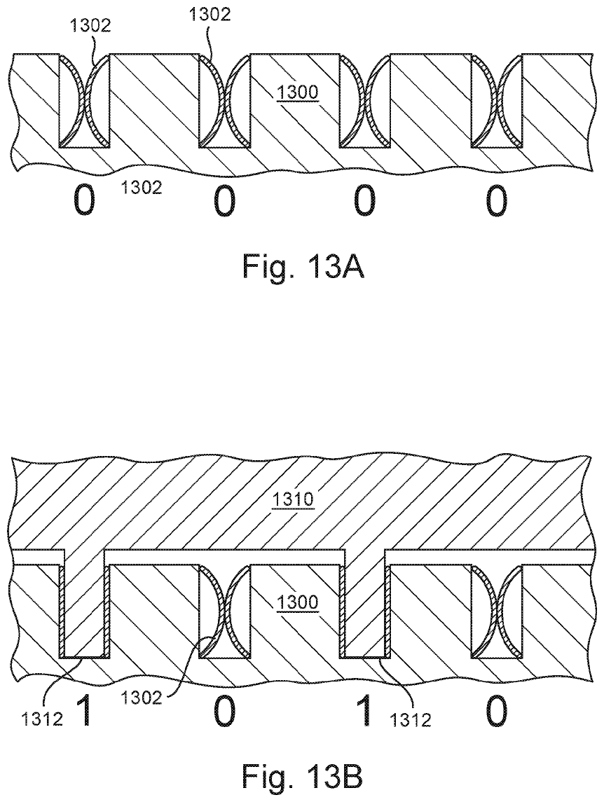

In some examples, a scent cartridge may also comprise a plurality of electrical contacts. The plurality of electrical contacts may be arranged on an outer surface of the cartridge, for example preferably on the base of the container, or around the lid portion. The scent delivery device may also comprise a plurality of corresponding electrical contacts, wherein the electrical contacts of the scent delivery device and the cartridge are configured to be electrically coupled when the scent cartridge is installed within the scent delivery device. As an example, FIG. 13A shows a cross section of a portion of a scent delivery device 1300 comprising a plurality of electrical contacts 1302. These contacts 1302 may be arranged on an internal surface of the device housing, within the cartridge receiving portion. Each electrical contact may represent one bit in a scent ID. In the uncoupled configuration shown in FIG. 13A, the device reads “0000” indicating that no scent cartridge is present.

As shown in FIG. 13B, electrical contacts 1312 of the scent cartridge 1310 may be configured to electrically contact at least a portion of the plurality of contacts 1302 in the scent delivery device 1300, for example when the cartridge is installed within the scent delivery device. Upon electrical connection between the cartridge contacts 1312 and the detection contacts 1302, the scent delivery device 1300 is configured to identify the scent cartridge 131 by reading each of the pins which together provide a binary scent ID (e.g. “1010”). The set of parameters for atomisation may be inferred by comparison of the binary scent ID “1010” with a lookup table or equivalent. The skilled person will understand that whilst only four electrical contacts 1302 and two corresponding pins 1312 are shown in FIGS. 13A and 13B, this is not intended to be limiting and any other number of electrical contacts may be used, for example, eight electrical contacts 1302 would permit eight-digit binary scent IDs to identify scent cartridges.

In the context of the present disclosure other examples and variations of the apparatus and methods described herein will be apparent to a person of skill in the art.

1. A replaceable scent cartridge for a scent delivery device, comprising:
a container configured to reversibly couple to a piezoelectric element;
a porous material arranged at least partially inside the container, wherein the porous material is configured to carry a scented media; and
wherein the porous material is configured to contact a surface of the piezoelectric element when the container is coupled to the piezoelectric element, the piezoelectric element being configured to atomise the scented media.

2. The replaceable scent cartridge of claim 1, further comprising the piezoelectric element.

3. The replaceable scent cartridge of any preceding claim wherein the container further comprises a biasing means contacting the porous material, wherein the biasing means is configured to bias the porous material towards the piezoelectric element.

4. The replacement scent cartridge of claim 3, wherein the biasing means is a spring.

5. The replaceable scent cartridge of any preceding claim wherein the container is configured to reversibly couple to a lid portion, wherein the piezoelectric element is arranged at least partially within the lid portion.

6. The replaceable scent cartridge of any preceding claim wherein the container comprises:
a reservoir configured to hold scented liquid;
wherein the porous material is arranged between the reservoir and the piezoelectric element, and wherein the porous material is configured to carry the scented liquid.

7. The replaceable scent cartridge of claim 6 wherein the porous material is arranged in a layer of porous material between the reservoir and the piezoelectric element.

8. The replaceable scent cartridge of claim 6 wherein the porous material comprises a wick element arranged between the reservoir and the piezoelectric element.

9. The replaceable scent cartridge of any preceding claim wherein the container comprises a cavity, the cavity consisting of the porous material soaked in scented liquid.

10. The replaceable scent cartridge of any preceding claim wherein the porous material is configured to transport scented media to the piezoelectric element by capillary action.

11. The replaceable scent cartridge of any preceding claim wherein the piezoelectric element is a microporous piezoelectric element.

12. The replaceable scent cartridge of any of claims 5 to 11 further comprising a lid configured to couple to the container, wherein the lid comprises the piezoelectric element.

13. The replaceable scent cartridge any preceding claim further comprising a nozzle configured to be in fluid communication with the piezoelectric element, wherein the nozzle is configured to provide an outlet for scented media atomised by the piezoelectric element.

14. The replaceable scent cartridge of claim 13 wherein the nozzle is configured to protrude from an outlet of a scent delivery device, when the replaceable scent cartridge is installed within said scent delivery device.

15. The replaceable scent cartridge of any of claims 2 to 14 further comprising at least one electrical contact, wherein the at least one electrical contact is configured electrically couple the piezoelectric element to an electrical contact of a scent delivery device when the replaceable scent cartridge is installed within said scent delivery device.

16. A resealable scent cartridge for a scent delivery device, comprising:
a container configured to hold a scented media, and
an impermeable membrane comprising at least one resealable aperture, wherein the impermeable membrane is configured to seal the resealable aperture in a closed configuration, and unseal the resealable aperture in an open configuration.

17. The resealable scent cartridge of claim 16 wherein the cartridge is configured to be biased into the open configuration by deforming the impermeable membrane.

18. The resealable scent cartridge of claim 17 wherein the impermeable membrane is configured to be deformed by deforming the container.

19. The resealable scent cartridge of any of claims 16 to 18 wherein the cartridge is configured to couple to a lid, and wherein the impermeable membrane is configured to be biased into the open configuration when the cartridge is coupled to the lid.

20. The resealable cartridge of claim 19 wherein the lid is configured to deform the container such that the impermeable membrane is deformed into the open configuration.

21. The resealable scent cartridge of claim 19 wherein the impermeable membrane is configured to be biased into the open configuration by a structure coupled to a surface of the lid.

22. The resealable scent cartridge of claim 21 wherein the structure is configured to extend through the at least one resealable aperture.

23. The resealable scent cartridge of any claims 16 to 22 wherein the lid comprises a piezoelectric element configured to atomise scented media.

24. The scent cartridge of any preceding claim further comprising the scented media, and a scent indication, wherein the scent indication comprises a set of parameters for atomisation of the scented media.

25. The scent cartridge of any preceding claim wherein the container further comprises a cleaning means, wherein the cleaning means is configured to clean the piezoelectric element when the container is coupled to the piezoelectric element.

26. The scent cartridge of any preceding claim further comprising a sensor configured to detect an indication of the amount of scented media within the container.

27. The scent cartridge of claim 26 wherein the sensor is configured to detect when the amount of scented media within the container is below a predetermined threshold.

28. The scent cartridge of claim 26 wherein the sensor is configured to detect concentration of the scented media released from the replaceable cartridge during atomisation.

29. A scent delivery device comprising:
a wearable housing configured to be worn by a user, wherein the housing comprises:
at least one outlet:
at least one scent cartridge of any preceding claim, in fluid communication with at least one outlet such that atomised scented media is configured to leave the housing via the outlet; and
a controller configured to control a piezoelectric element to control atomisation of scented media held by said scent cartridge.

30. A scent delivery device comprising:
a wearable housing configured to be worn by a user, the housing comprising at least one outlet; and
at least one atomiser element configured to couple to a scent cartridge, wherein the atomiser element is configured to atomise scented media held by the scent cartridge; and
wherein each atomiser element is in fluid communication with at least one outlet such that the atomised scented media is configured to leave the housing via the outlet.

31. A scent delivery device comprising:
a wearable housing configured to be worn by a user, wherein the housing comprises:
at least one outlet; and
at least one cartridge receiving portion configured to receive a scent cartridge, wherein the at least one cartridge receiving portion comprises an electrical contact configured to couple to an atomiser element housed within said scent cartridge when said scent cartridge is arranged within the cartridge receiving portion;
and a controller, coupled to the electrical contact, the controller being configured to control said atomiser element of said scent cartridge, via the electrical contact, to control atomisation of scented media held by said scent cartridge;
wherein the at least one cartridge receiving portion is in fluid communication with at least one outlet such that scented media atomised by said scent cartridge is configured to leave the housing via the outlet.

32. The scent delivery device of claim 29 to 31 wherein the wearable housing comprises a neck-mountable housing configured to be worn at least partially around the neck of a user.

33. The scent delivery device of any of claims 29 to 32 wherein the wearable housing is configured to attach to an item of clothing worn by the user.

34. The scent delivery device of any of claims 29 to 33 wherein the housing further comprises at least one flexible portion configured to be arranged adjacent to the back of the neck of a user.

35. The scent delivery device of any of claims 29 to 34 wherein the housing comprises a moveable portion configured to be repositioned relative to the user, wherein the outlet is arranged within the moveable portion.

36. The scent delivery device of any of claims 29 to 35 further comprising at least one fan, wherein the fan is configured to clear the atomised scented media.

37. The scent delivery device of any of claims 29 to 36 wherein the housing comprises an arched shape configured to be worn around the neck of a user.

38. The scent delivery device of claim 37 comprising an outlet at each end of the arched shape housing.

39. The scent delivery device of any of claims 29 to 38 wherein each atomiser element is configured to be independently controlled.

40. The scent delivery device of claim 39 further comprising an inertial measurement unit, wherein each atomiser element is configured to be controlled based on an indication determined by the inertial measurement unit.

41. The scent delivery device of any of claims 39 to 40 further comprising a means for determining location, wherein each atomiser element is configured to be controlled based on an indication determined by the means for determining location.

42. A scent delivery device for treatment or monitoring of anosmia, comprising:
a housing comprising an outlet;
an atomiser element configured to couple to a scent cartridge, wherein the atomiser element is configured to atomise scented media held by the scent cartridge; wherein the atomiser element is in fluid communication with the outlet such that atomised scented media is configured to leave the housing via the outlet; and
a sensor configured to sense concentration of the scented media released from the cartridge during atomisation.

43. The scent delivery device of claim 42 wherein the sensor is arranged within the housing such that the sensor is configured to sense concentration of the scented media released at the outlet.

44. The scent delivery device of any claims 42 to 43 further comprising a processor configured to control operation of the atomiser element, wherein the processor is configured to control the atomiser element to vary the concentration of the scented media released from the scent cartridge during atomisation across a time window.

45. The scent delivery device of claim 44 wherein the atomiser element comprises a piezoelectric element, and wherein the processor is configured to control the atomiser element to vary the concentration of the scented media released from the cartridge during atomisation by controlling at least one of (i) the duration of vibration, and (ii) the frequency of vibration of the piezoelectric element.

46. The scent delivery device of any of claims 42 to 45 further comprising a communications interface configured to send indications of the concentration sensed by the sensor to a remote device.

47. The scent delivery device of any claims 42 to 46 further comprising a user input interface configured to receive user input relating to the user's perception of smell of the atomised scented media.

48. The scent delivery device of any of claims 29 to 47 further comprising at least one cleaning means, wherein each cleaning means is configured to clean the at least one atomiser element.

49. The scent delivery device of any claims 29 to 48 further comprising at least one scent cartridge configured to hold scented media, each scent cartridge being coupled to one of the at least one atomiser elements;
wherein each scent cartridge is in fluid communication with at least one outlet such that the atomised scented media is configured to leave the housing via the outlet.

50. The scent delivery device of claim 49, wherein the at least one cartridge is configured to be replaceable, the scent delivery device further comprising at least one sensor, each sensor being configured to detect an indication of the amount of scented media within at least one replaceable cartridge arranged within the housing.

51. The scent delivery device of claim 50 wherein the sensor is configured to detect when the amount of scented media within the replaceable cartridge is below a predetermined threshold.

52. The scent delivery device of claim 50 wherein the sensor is configured to detect concentration of the scented media released from the replaceable cartridge during atomisation.

53. The scent delivery device of any of claims 29 to 52 further comprising at least one vent configured to draw air into the housing.

54. The scent delivery device of any of claims 49 to 53 wherein each cartridge is configured to be replaceable, and wherein each replaceable cartridge comprises a scent indication, wherein the scent indication comprises a set of parameters for atomisation of the scented media, and wherein the scent delivery device further comprises:
a detection means configured to detect the scent indication; and
a processor configured to control the atomiser element based on the detected scented indication.
